(19) United States
(12) Patent Application Publication
(10) Pub. No.: US 2013/0045933 A1
(43) Pub. Date: Feb. 21, 2013

(54) ALOE-EMODIN DERIVATIVES AND USE THEREOF FOR THE TREATMENT OF CANCER

(71) Applicants: Micha Fridman, Qiryat Bialik (IL); Elinor Briner-Goldstein, Ness-Ziona (IL); Zoharia Evron, Hod Hasharon (IL); Michael Frenkel, Pardes-Hanna (IL); Ester Flescher, Hod Hasharon (IL)

(72) Inventors: Micha Fridman, Qiryat Bialik (IL); Eliezer Flescher, Hod Hasharon (IL); Elinor Briner-Goldstein, Ness-Ziona (IL); Zoharia Evron, Hod Hasharon (IL); Michael Frenkel, Pardes-Hanna (IL)

(73) Assignee: RAMOT AT TEL-AVIV UNIVERSITY LTD., Tel Aviv (IL)

(21) Appl. No.: 13/574,558

(22) PCT Filed: Jan. 20, 2011
(86) PCT No.: PCT/IL11/00067

§ 371 Date: Aug. 1, 2012

Related U.S. Application Data
(60) Provisional application No. 61/296,915, filed on Jan. 21, 2010.

(51) Int. Cl. C07H 15/244; A61P 35/04; A61P 35/02; A61K 31/704; A61P 35/00

(52) U.S. Cl. 514/25; 536/17.2

(57) ABSTRACT

The present invention relates to anthracycline derivatives that are based on an Aloe-emodin (AE) backbone attached to a glycoside (an amino sugar or amino carba-sugar). These derivatives are useful as chemotherapeutic agents. Advantageously, these derivatives are potent cytotoxic agents against a variety of anthracycline-resistant tumors. In addition, they may have reduced cardiotoxicity. As such, the novel compounds of the invention offer an advantage over currently available drugs. The present invention further relates to methods for preparing the novel Aloe-Emodin Glycoside (AEG) based derivatives, pharmaceutical compositions including such compounds, and methods of using these compounds and compositions, especially as chemotherapeutic agents for prevention and treatment of cancers.

### FIELD OF THE INVENTION

The present invention relates to novel Aloe-emodin (AE) based derivatives, methods for their preparation, pharmaceutical compositions including such compounds, and methods of using these compounds and compositions, especially as chemotherapeutic agents for prevention and treatment of cancers, in particular cancers that are resistant to anthracyclines such as doxorubicin.

### BACKGROUND OF THE INVENTION

Anthracyclines (FIG. 1) are anti-tumor agents that act by interfering with DNA synthesis which then leads to inhibition of DNA replication and cell division.^{[1-3]} Two mechanisms have been suggested to explain how anthracyclines exert their antitumor activity. Intercalation of the anthraquinone part of the molecule between the DNA base pairs, and the interactions of the sugar side chain with residues in the minor groove of the double helix contribute to DNA binding affinity.^{[4]} DNA binding of anthracyclines interferes with DNA replication which directly affects malignant cells.

Other scientific observations indicate that anthracycline anti-tumor activity may be ascribed to the ability of anthracyclines to interfere with DNA topoisomerase II function.^{[5]} The carbohydrate rings of anthracyclines were proposed to participate in the stabilization of a ternary complex (DNA-drug-topoisomerase II).^{[6,7]} Anthracyclines stabilize a transient DNA-topoisomerase II complex in which DNA strands are cut and covalently linked to the enzyme subunits.^{[8,9]} Topoisomerase II activity is required for DNA replication and as such, the ternary complex (DNA-drug-topoisomerase II) inhibits DNA replication and therefore cell division.

### Cardio-Toxicity of Anthracyclines

Cardiomyopathy is a severe clinical side effect of anthracycline administration that seriously limits the therapeutic window of these compounds.^{[10]} Intensive research effort has focused on studying the molecular mechanisms of the severe toxic side effects of anthracyclines based chemotherapeutic treatments. For example, a study of the metabolism of doxorubicin and its effect on human myocardium cells identified the formation of toxic metabolites (FIG. 2).^{[10]} In fractions obtained from cardiac cell cytosol, alcohol metabolites such as doxorubicinol, deoxy-rubicinol and rubicinol, were recovered.

The cleavage of the glycosidic bond is a major pathway in the metabolism of anthracyclines in mammalian cell systems.^{[11]} The reaction is catalyzed by NADPH-cytochrome P450 reductase, xanthine oxidase, and DT-diaphorase. Preventing the deglycosylation of anthracyclines inside mammalian cells is therefore suggested as a rational direction to circumvent the major cause for the severe toxic side effects of anthracyclines.

It has been observed that structural differences in the carbohydrate structure of anthracyclines lead to a decrease in the production of toxic metabolites. For example, doxorubicin differs from epirubicin in a single carbohydrate stereo-center (C-4 alcohol of the carbohydrate (FIG. 3). Both these anthracyclines have marked anti-tumor activities. However, reduction by NADPH dependant carbonyl reductase was observed to be much less significant for epirubicin than for doxorubicin.^{[12]} Indeed, epirubicin was found to exhibit significantly less endomyocardial damage than doxorubicin. A similar decrease in toxicity was observed with MEN 10755 (FIG. 3), a novel anthracycline with preclinical evidence of reduced cardio-toxicity.^{[13]}

### Anthracycline Drug Resistance

Another major limitation on the clinical use of anthracylines results from the emergence of tumor cells with resistance to treatment by these chemotherapeutic agents.^{[14-15]} To date, well over 2000 analogues of anthracyclines have been synthesized in search for compounds with improved clinical performance, yet only very few demonstrated improved anticancer activity and became clinically used.^{[16]} Synthetic efforts focused on methods to vary the anthraquinone and/or sugar scaffolds of the parent anthracylines.^{[17-20]}

Aloe-emodin (AE) is a hydroxyanthraquinone present in Aloe vera leaves.^{[21-23]} The parent molecule suffers from the disadvantage of being hardly soluble in water and in physiological solutions, while it is soluble only in hot alcohols, ethers, benzene and in water alkalinized with ammonia or acidified by sulfuric acid. Therefore, from a pharmaceutical point of view, these characteristics make the parent molecule problematic for use in therapeutic treatments.

WO 02/090313 discloses AE derivatives bearing a positive or negative charge at position 3′, and their use in the treatment of neoplasias. These derivatives are described as exhibiting improved solubility, while maintaining the same biological activity as AE and being potential AE pro-drugs. One specific such derivative is an AE acetal with the amino sugar daunosamine.

There is an unmet medical need for anthracycline-based chemotherapeutic agents that have potent anti-tumor activity on the one hand, while having reduced cardio-toxicity on the other, and/or that are effective at treating anthracycline resistant tumors.

### SUMMARY OF THE INVENTION

The present invention relates to anthracycline derivatives that are based on an Aloe-emodin backbone attached to an amino sugar or amino carba-sugar. These novel derivatives, designated herein Aloe-Emodin Glycoside (AEG) derivatives, are useful as chemotherapeutic agents. The present invention further relates to methods for preparing the novel AE based derivatives, pharmaceutical compositions including such compounds, and methods of using these compounds and compositions, especially as chemotherapeutic agents for prevention and treatment of cancers.

The widespread clinical use of anthracyclines as anticancer agents and the very high success rate of these drugs in the treatment of various cancers puts such compounds on the front lines of cancer treatment options. However, drug resistance to anthracycline chemotherapeutic agents has become a major impediment on their clinical use. The present invention introduces a new family of synthetic anthracyclines that are, in some embodiments, active against a variety of cancers that are highly resistant to anthracycline chemotherapy, while maintaining DNA binding properties and cytotoxic activity. In particular, it has unexpectedly been discovered that these AEG derivatives are at least two orders of magnitude more potent than Doxorubicin and Aloe-Emodin against various doxorubicin-resistant tumors. As such, the compounds of the invention offer significant advantages over conventional anthracycline-based chemotherapeutic agents.

Moreover, severe cardio-toxicity is a major side effect of such drugs, which dramatically limits their use in the clinic. The cardio-toxicity caused by anthracyclines is believed to result from their metabolism in mammalian cells. Highly toxic metabolites are formed by removal of the carbohydrate scaffold from the anthracycline through reductive de-glycosidation in mammalian cells. The present invention introduces a new family of synthetic anthracyclines that are, in some embodiments, chemically resistant to reductive de-glycosidation, while maintaining DNA binding properties and cytotoxic activity. As such, these novel compounds are useful as chemotherapeutic agents that display anti-tumor activity. In some embodiments, the derivatives display reduced cardio-toxicity, thus offering an advantage over conventional anthracycline therapy.

In one embodiment, the present invention relates to a compound represented by the structure of formula (I)

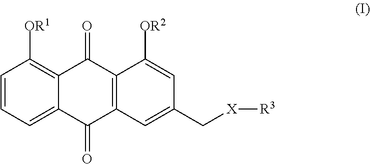

wherein

R¹ and R² are independently H or a C1-C4 alkyl;

R³ is an amino sugar or an amino carba-sugar; and

X is O or S,

with the proviso that when both R¹ and R² are H and X is O, R³ is not

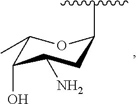

including salts, solvates, polymorphs, optical isomers, geometrical isomers, enantiomers, diastereomers, and mixtures thereof.

In one embodiment, R¹ and R² are H, thus representing the parent AE backbone. In some embodiments, the compound is a methylated or a dimethylated derivative of Aloe-emodin. Thus, in one embodiment, R¹ is H and R² is CH₃. In another embodiment, R¹ is CH₃ and R² is H. In yet another embodiment, R¹ and R² are both CH₃. Each possibility represents a separate embodiment of the present invention.

In one embodiment, X is O, and the bond between the AE and the amino sugar is a glycosidic bond, which can be an alpha (α) glycosidic bond or a beta (β) glycosidic bond. In another embodiment, X is S, and the bond between the Aloe-emodin and the amino sugar is a thio-glycosidic bond, which can be an alpha (α) thio-glycosidic bond or a beta (β) thio-glycosidic bond. Each possibility represents a separate embodiment of the present invention.

In further embodiments, the sugar in the compound of formula (I) is an amino sugar (i.e., R³ in compound (I) is an amino sugar), preferably in the form of an acetal or a thioacetal of an amino sugar. The amino sugar may be a 2-deoxy amino sugar, a 3-deoxy amino sugar, a 6-deoxy amino sugar, a 2,3-dideoxy amino sugar, a 2,6-dideoxy amino sugar, a 3,6-dideoxy amino sugar or a 2,3,6-trideoxy amino sugar. Each possibility represents a separate embodiment of the present invention. In one currently preferred embodiment, the amino group is at the C-3 position, thus representing a 3-amino sugar. The 3-amino sugar may be a 2-deoxy-3-amino sugar, a 3-deoxy-3-amino sugar, a 6-deoxy-3-amino sugar, a 2,3-dideoxy-3-amino sugar, a 2,6-dideoxy 3-amino sugar, a 3,6-dideoxy-3-amino sugar, or more preferably a 2,3,6-trideoxy-3-amino sugar. 4-deoxy amino sugars, including 2,4-deoxy, 3,4-deoxy, 4,6-deoxy, 2,3,4-trideoxy, 3,4,6-trideoxy, 2,4,6-trideoxy and 2,3,4,6-tetradeoxy amino sugars are also contemplated. Each possibility represents a separate embodiment of the present invention.

In yet other embodiments, the amino sugar is in the form of a pyranoside, which can be a pentose pyranoside or a hexose pyranoside, preferably a hexose pyranoside. In one embodiment, the amino sugar is a 2-deoxypyranose form of an aldopentose. In other embodiments, the amino sugar is a 2-deoxy pyranose form of an aldohexose. Other possibilities include, but are not limited to a 3-deoxypyranose form of an aldopentose, a 2,3-dideoxypyranose form of an aldopentose, a 3-aminopyranose form of an aldopentose, a 2-deoxy-3-aminopyranose form of an aldopentose, a 3-deoxy-3-aminopyranose form of an aldopentose, a 2,3-dideoxy-3-aminopyranose form of an aldopentose, a 3-deoxy pyranose form of an aldohexose, a 6-deoxy pyranose form of an aldohexose, a 2,3-dideoxy pyranose form of an aldohexose, a 2,6-dideoxy pyranose form of an aldohexose, a 3,6-dideoxy pyranose form of an aldohexose, a 2,3,6-trideoxy pyranose form of an aldohexose, a 3-aminopyranose form of an aldohexose, a 2-deoxy-3-amino-pyranose form of an aldohexose, a 3-deoxy-3-amino-pyranose form of an aldohexose, a -6-deoxy-3-amino-pyranose form of an aldohexose, a 2,3-dideoxy-3-amino-pyranose form of an aldohexose, a 2,6-dideoxy-3-amino-pyranose form of an aldohexose, a 3,6-dideoxy-3-amino-pyranose form of an aldohexose, and a 2,3,6-trideoxy-3-amino-pyranose form of an aldohexose. 4-deoxy amino pyranose sugars of aldopentoses and aldohexoses, including 2,4-deoxy, 3,4-deoxy, 4,6-deoxy, 2,3,4-trideoxy, 3,4,6-trideoxy, 2,4,6-trideoxy and 2,3,4,6-tetradeoxy pyranose amino sugars are also contemplated. Each possibility represents a separate embodiment of the present invention.

The amine group of the amino sugar can be in the axial or equatorial position, preferably in the equatorial. The glycosidic bond can be an α-glycosidic bond or a an β-glycosidic bond. In one embodiment, the amine or the amino sugar is at the C-3 equatorial position and the amino sugar is linked through an α-glycosidic bond. In another embodiment, the amine or the amino sugar is at the C-3 equatorial position and the amino sugar is linked through a β-glycosidic bond. In another embodiment, the amine or the amino sugar is at the C-3 axial position and the amino sugar is linked through an α-glycosidic bond. In another embodiment, the amine or the amino sugar is at the C-3 axial position and the amino sugar is linked through a β-glycosidic bond. Each possibility represents a separate embodiment of the present invention.

In some representative embodiments, the amino sugar is a pentose pyranoside selected from the group consisting of:

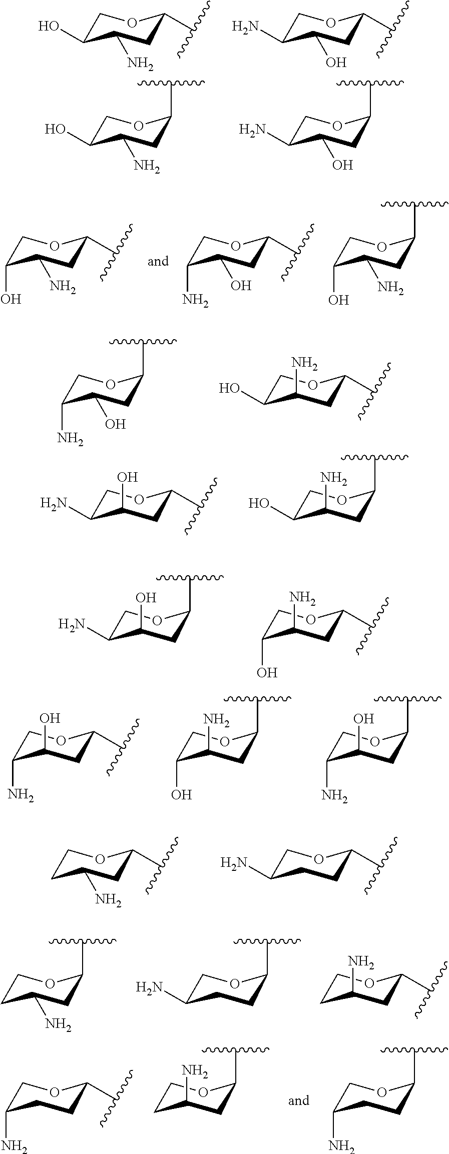

Each one of the aforementioned possibilities represents a separate embodiment of the present invention.

In other representative embodiments, the amino sugar is a hexose pyranoside selected from the group consisting of:

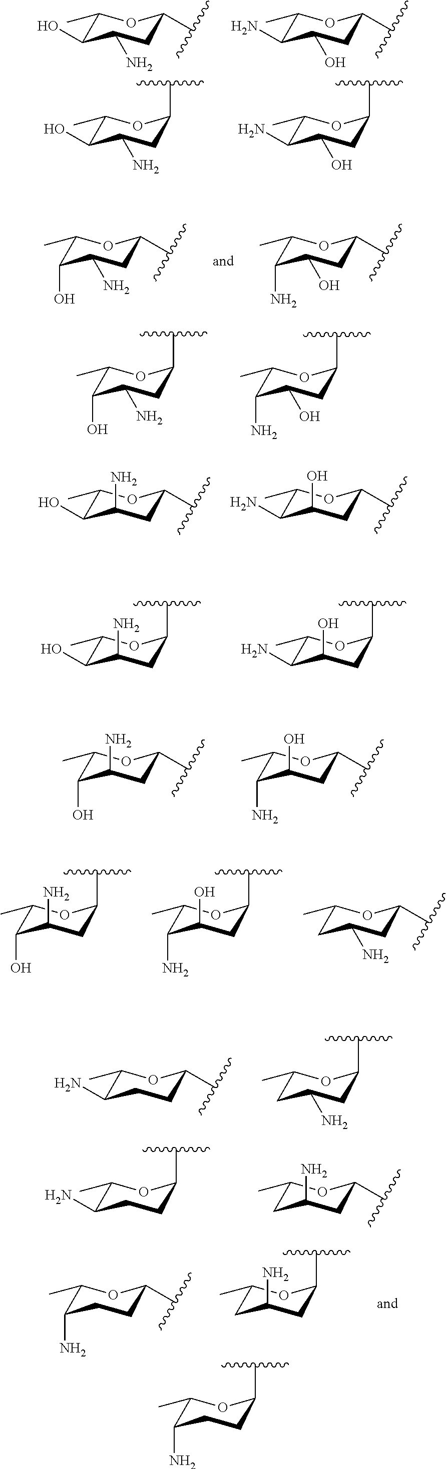

Each one of the aforementioned possibilities represents a separate embodiment of the present invention.

In some preferred embodiments, the amino-sugar is selected from the group consisting

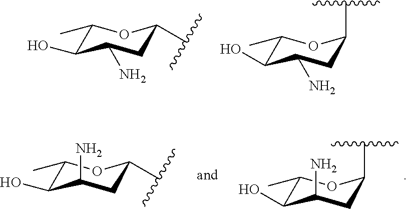

In one currently preferred embodiment, the amino sugar is represented by the structure:

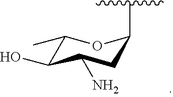

In some specific embodiments, the compound of formula (I) is represented by the structure:

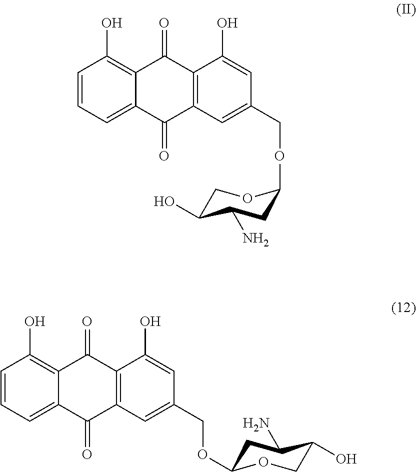

In other specific embodiments, the compound of formula (I) is represented by the structure:

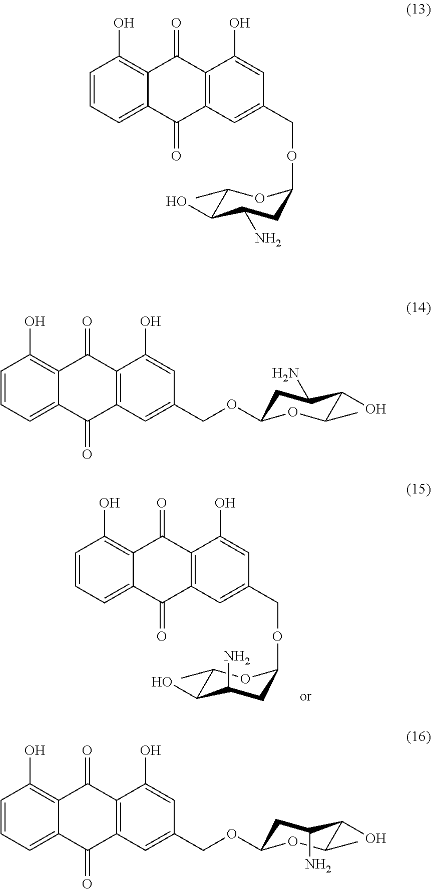

In further embodiments, the sugar in the compound of formula (I) is an amino carba-sugar (i.e., R³ in compound (I) is an amino carba-sugar), preferably in the form of an acetal or a thioacetal of an amino carba-sugar. The amino carba-sugar may be is a 2-deoxy amino carba-sugar, a 3-deoxy amino carba-sugar, a 6-deoxy amino carba-sugar, a 2,3-dideoxy amino carba-sugar, a 2,6-dideoxy amino carba-sugar, a 3,6-dideoxy amino carba-sugar or a 2,3,6-trideoxy amino carba-sugar. Each possibility represents a separate embodiment of the present invention. In one currently preferred embodiment, the amino group is at the C-3 position, thus representing a 3-amino carba-sugar. The 3-amino carba-sugar may be a 3-amino carba-sugar, a 2-deoxy-3-amino carba-sugar, a 3-deoxy-3-amino carba-sugar, a 6-deoxy-3-amino carba-sugar, a 2,3-dideoxy-3-amino carba-sugar, a 2,6-dideoxy 3-amino carba-sugar, a 3,6-dideoxy-3-amino carba-sugar, or a 2,3,6-trideoxy-3-amino carba-sugar. 4-deoxy amino carba-sugars, including 2,4-deoxy, 3,4-deoxy, 4,6-deoxy, 2,3,4-trideoxy, 3,4,6-trideoxy, 2,4,6-trideoxy and 2,3,4,6-tetradeoxy amino carba-sugars are also contemplated. Each possibility represents a separate embodiment of the present invention.

The amine group of the amino carba-sugar can be in the axial or equatorial position, preferably in the equatorial. Each possibility represents a separate embodiment of the present invention.

In some representative embodiments, the amino carba-sugar is selected from the group consisting of:

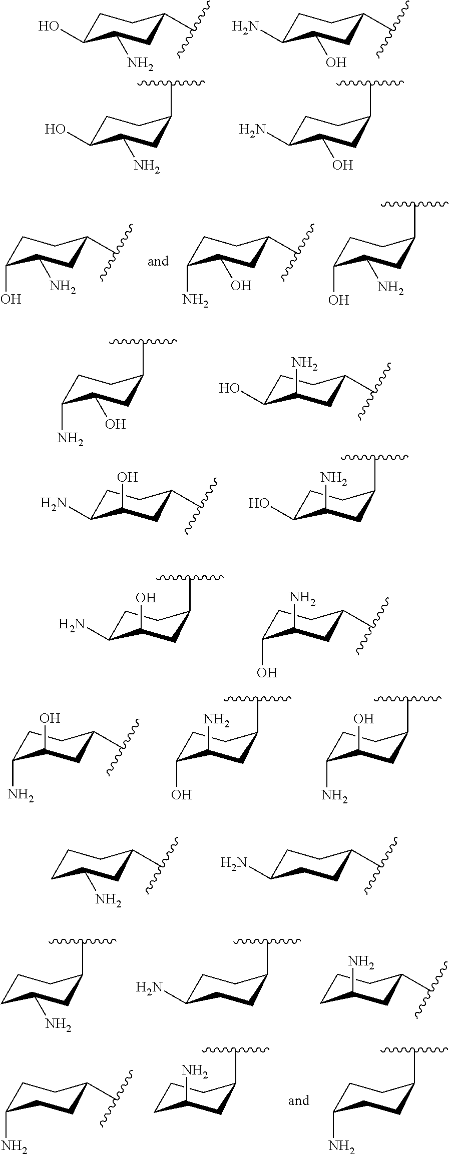

Each one of the aforementioned possibilities represents a separate embodiment of the present invention.

In other representative embodiments, the amino carba-sugar is selected from the group consisting of:

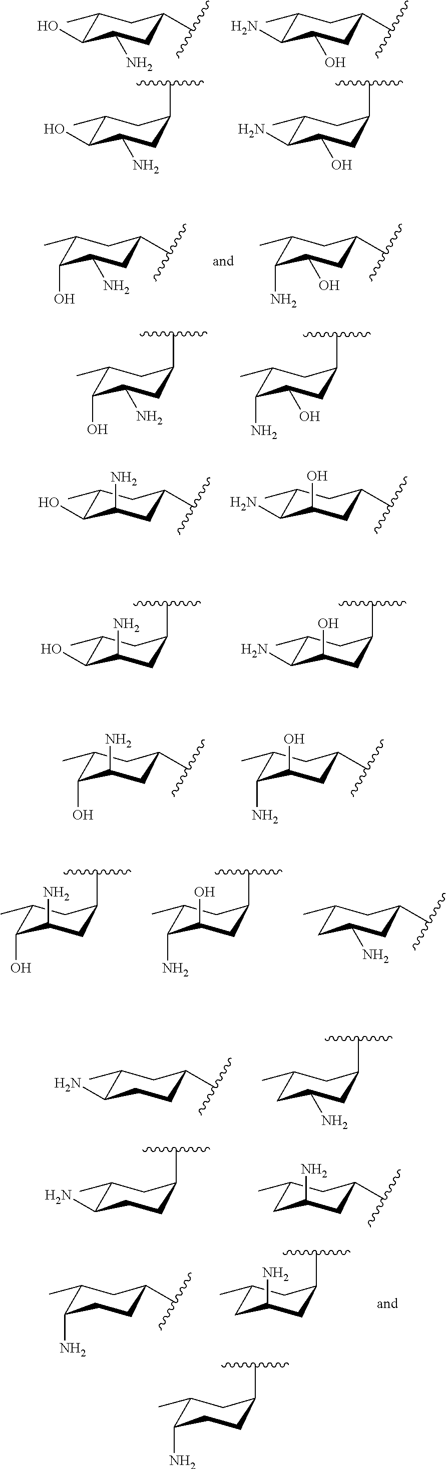

Each one of the aforementioned possibilities represents a separate embodiment of the present invention.

In a specific embodiment, the compound of formula (I) compound is represented by the structure:

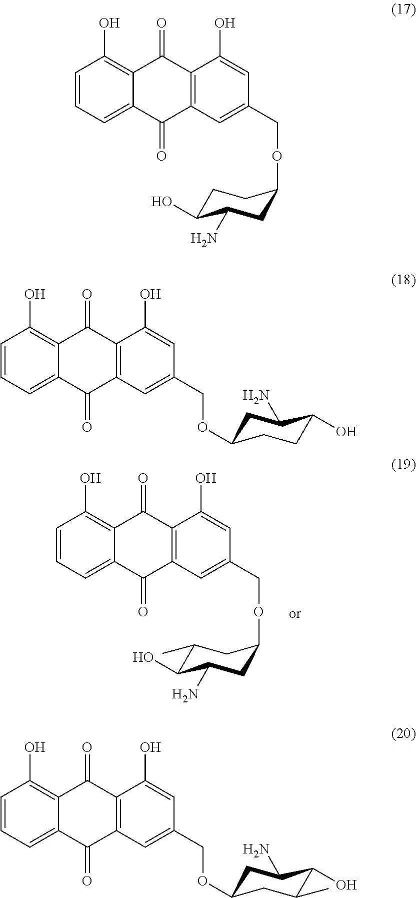

The amino-sugar moiety in the compounds of the invention may be a D-sugar or an L-sugar. Each possibility represents a separate embodiment of the present invention.

In further embodiments, the present invention relates to a pharmaceutical composition comprising a compound of formula (I), or a compound of formula (II), (12), (13), (14), (16), (16), (17), (18), (19) or (20) and a pharmaceutically acceptable excipient. The composition can be in a form suitable for oral administration, intravenous administration by injection, topical administration, administration by inhalation, or administration via a suppository. In a currently preferred embodiment, the pharmaceutical composition is in a form suitable for oral administration.

The present invention further provides methods for inhibiting cancer cell proliferation, comprising contacting the cancer cells with a therapeutically effective amount of a compound of formula (I) or a compound of formula (II), (12), (13), (14), (16), (16), (17), (18), (19) or (20). In another embodiment, the present invention provides a method of treating cancer in a subject in need thereof, comprising the step of administering to the subject a therapeutically effective amount of formula (I) or a compound of formula (II), (12), (13), (14), (16), (16), (17), (18), (19) or (20). In other embodiments, the present invention relates to the use of a compound of formula (I) or a compound of formula (II), (12), (13), (14), (16), (16), (17), (18), (19) or (20) for treating cancer. In other embodiments, the present invention relates to the use of a compound of formula (I) or a compound of formula (II), (12), (13), (14), (16), (16), (17), (18), (19) or (20) for the preparation of a medicament for treating cancer. In other embodiments, the present invention relates to a compound of formula (I) or a compound of formula (II), (12), (13), (14), (16), (16), (17), (18), (19) or (20) for use in the treatment of cancer. In one embodiment, the cancer is a mammalian cancer, e.g., a human cancer.

In some embodiments, the cancer is selected from the group consisting of: a sarcoma, melanoma, a carcinoma, a leukemia (e.g., T-cell leukemia), adenocarcinoma (e.g., colon adenocarcinoma) and fibrosarcoma, as well as metastases of all the above. Each possibility represents a separate embodiment of the present invention.

In other embodiments, the cancer is selected from the group consisting of lymphoproliferative disorders, breast cancer, ovarian cancer, prostate cancer, cervical cancer, endometrial cancer, bone cancer, liver cancer, stomach cancer, colon cancer, pancreatic cancer, cancer of the thyroid, head and neck cancer, cancer of the central nervous system, cancer of the peripheral nervous system, skin cancer and kidney cancer, hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, squamous cell carcinoma, basal cell carcinoma, adenocarcinoma (well differentiated, moderately differentiated, poorly differentiated or undifferentiated), renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma and non-Hodgkin's lymphoma, as well as metastases of all of the above. Each possibility represents a separate embodiment of the present invention.

In some embodiments, wherein the cancer is characterized by resistance to anthracycline chemotherapeutic agents such as doxorubicin. Non-limiting examples of anthracycline cancers include lymphoproliferative disorders, breast cancer, ovarian cancer, prostate cancer, colon cancer, pancreatic cancer, sarcoma, fibrosarcoma, melanoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma and non-Hodgkin's lymphoma, as well as metastases of all of the above. Each possibility represents a separate embodiment of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1: Structures of common anthracyclines

FIG. 2: Doxorubicin metabolism in human cardiac cytosol

FIG. 3: Doxorubicin and epirubicin structures

FIG. 4: Cytotoxicity of doxorubicin (Dox), Aloe-Emodin (Alo) and Alo derivatives (E1 and E2, also referred to as compounds 11 and 12, respectively) towards cancer cells Molt4 (FIG. 4A); B16 (FIG. 4B); HCT116 (FIG. 4C); and MCA 105 (FIG. 4D).

FIG. 5: Cytotoxicity of doxorubicin (Dox) (FIG. 5A), Aloe-Emodin (AE) (FIG. 5B) and AE derivatives AEGs 13-16 (FIG. 5C-5F) towards cancer cells Molt4.

FIG. 6: Cytotoxicity of doxorubicin (Dox) (FIG. 6A), Aloe-Emodin (AE) (FIG. 6B) and AE derivatives AEGs 13-16 (FIG. 6C-6F) towards DOX-resistant ovarian cancer cells OVAR-3.

FIG. 7: Cytotoxicity of doxorubicin (Dox) (FIG. 7A), Aloe-Emodin (AE) (FIG. 7B) and AE derivatives AEGs 13-16 (FIG. 7C-7F) towards DOX-resistant breast cancer cells MCF-7.

FIG. 8: Cytotoxicity of doxorubicin (Dox) (FIG. 8A), Aloe-Emodin (AE) (FIG. 8B) and AE derivatives AEGs 13-16 (FIG. 8C-8F) towards DOX-resistant ovarian cancer cells NAR.

FIG. 9: Light microscopy (x400) pictures of MCF-7 cells: Cell cultures were incubated for 24 hours. (a) Untreated control cells (b) AE 20 μM (c), Dox 20 μM, (d) AEG 13 20 μM.

FIG. 10. Supercoiled plasmid DNA unwinding gel experiment: (1) Untreated DNA, (2) AE 200 μM, (3) AEG 13 200 μM, (4) AEG 13 20 μM, (5) AEG I4 200 μM, (6) AEG 14 20 μM, (7) AEG 15 200 μM, (8) AEG 15 20 μM, (9) AEG 16 200 μM, (10) AEG 16 20 μM, (11) DOX 200 μM, (12) DOX 20 μM.

FIG. 11: Confocal microscopy images of DOX or AEG 13 pre-incubated NAR cells: Cells were pre-incubated with 5 μM of DOX (a-c) or 5 μM of AEG 13 (d-f). The plasma membrane was stained with carbocyanine tracer DiD (DiIC₁₈ (5)-DS).

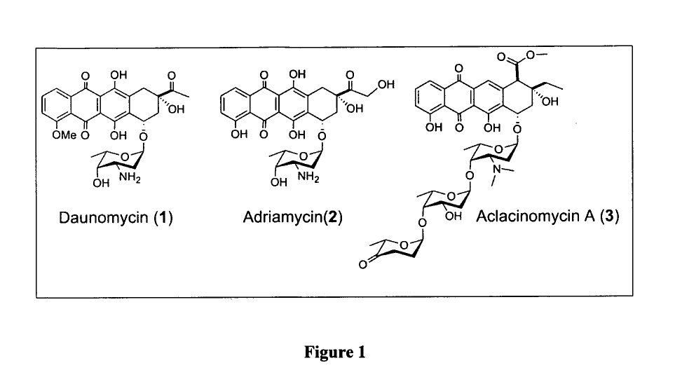

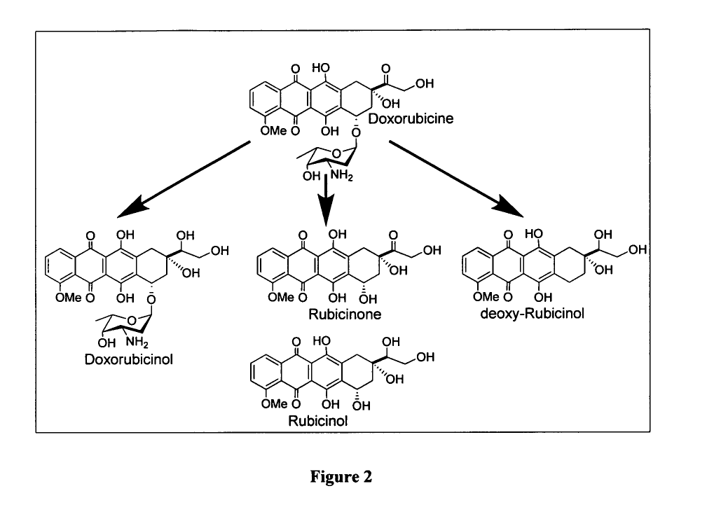

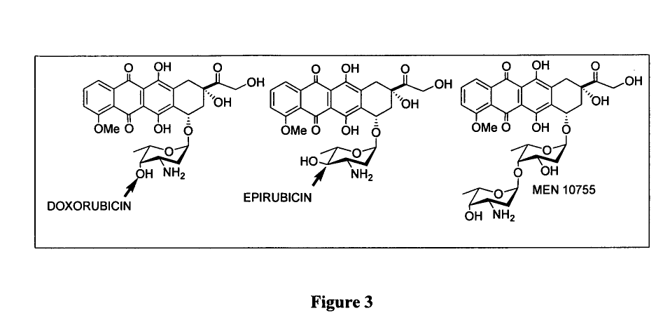

### DETAILED DESCRIPTION OF THE INVENTION

The present invention relates to anthracycline derivatives that are based on an Aloe-emodin backbone attached to an amino sugar or amino carba-sugar. These novel derivatives have reduced anthracycline-related cardio-toxicity, and are useful as chemotherapeutic agents. In particular, these agents exhibit unexpected cytotoxic potency against a variety of cancer cells that are resistant to anthracycline chemotherapeutic agents such as doxorubicin. As such, these agents present a novel strategy to target anthracycline resistant tumors.

### Compounds

The compounds of the present invention are generally represented by the structure of formula (I):

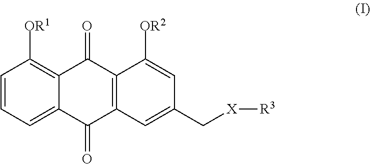

wherein

R¹ and R² are independently H or a C1-C4 alkyl;

R³ is an amino sugar or an amino carba-sugar; and

X is O or S,

with the proviso that when both R¹ and R² are H and X is O, R³ is not

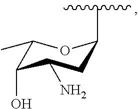

including salts, solvates, polymorphs, optical isomers, geometrical isomers, enantiomers, diastereomers, and mixtures thereof.

The term “C₁-C₄ alkyl” used herein alone or as part of another group denotes linear and branched, saturated or unsaturated (e.g., alkenyl, alkynyl) groups, the latter only when the number of carbon atoms in the alkyl chain is greater than or equal to two, and can contain mixed structures. Examples of saturated alkyl groups include but are not limited to methyl, ethyl, n-propyl, isopropyl, n-butyl, iso-butyl, sec-butyl and tert-butyl. Examples of alkenyl groups include vinyl, allyl and the like. Examples of alkynyl groups include ethynyl, propynyl and the like. Similarly, the term “C₁ to C₄ alkylene” denotes a bivalent radicals of 1 to 4 carbons.

The C₁ to C₄ alkyl group can be unsubstituted, or substituted with one or more substituents selected from the group consisting of halogen, hydroxy, alkoxy, aryloxy, alkylaryloxy, heteroaryloxy, oxo, cycloalkyl, phenyl, heteroaryl, heterocyclyl, naphthyl, amino, alkylamino, arylamino, heteroarylamino, dialkylamino, diarylamino, alkylarylamino, alkylheteroarylamino, arylheteroarylamino, acyl, acyloxy, nitro, carboxy, carbamoyl, carboxamide, cyano, sulfonyl, sulfonylamino, sulfinyl, sulfinylamino, thiol, C₁ to C₁₀ alkylthio arylthio, or C₁ to C₁₀ alkylsulfonyl groups. Any substituent can be unsubstituted or further substituted with any one of these aforementioned substituents.

All stereoisomers of the above compounds are contemplated, either in admixture or in pure or substantially pure form. The Aloe-emodin sugar derivatives of the present invention can have asymmetric centers at any of the atoms. Consequently, the compounds can exist in enantiomeric or diastereomeric forms or in mixtures thereof. The present invention contemplates the use of any racemates (i.e. mixtures containing equal amounts of each enantiomers), enantiomerically enriched mixtures (i.e., mixtures enriched for one enantiomer), pure enantiomers or diastereomers, or any mixtures thereof. The chiral centers can be designated as R or S or R,S or d,D, l,L or d,l, D,L. The sugar residues include residues of D-sugars, L-sugars, or racemic derivatives of sugars.

One or more of the compounds of the invention, may be present as a salt. The term “salt” encompasses both basic and acid addition salts, including but not limited to carboxylate salts or salts with amine nitrogens, and include salts formed with the organic and inorganic anions and cations discussed below. Furthermore, the term includes salts that form by standard acid-base reactions with basic groups (such as amino groups) and organic or inorganic acids. Such acids include, but are not limited to, hydrochloric, hydrofluoric, trifluoroacetic, sulfuric, phosphoric, acetic, succinic, citric, lactic, maleic, fumaric, palmitic, cholic, pamoic, mucic, D-glutamic, D-camphoric, glutaric, phthalic, tartaric, lauric, stearic, salicylic, methanesulfonic, benzenesulfonic, sorbic, picric, benzoic, cinnamic, and like acids.

The term “organic or inorganic cation” refers to counter-ions for the carboxylate anion of a carboxylate salt. The counter-ions are chosen from the alkali and alkaline earth metals, (such as lithium, sodium, potassium, barium, aluminum and calcium); ammonium and mono-, di- and tri-alkyl amines such as trimethylamine, cyclohexylamine; and the organic cations, such as dibenzylammonium, benzylammonium, 2-hydroxyethylammonium, bis(2-hydroxyethyl)ammonium, phenylethylbenzylammonium, dibenzylethylenediammonium, and like cations. See, for example, “Pharmaceutical Salts,” Berge et al., J. Pharm. Sci., 66:1-19 (1977), which is incorporated herein by reference. Other cations encompassed by the above term include the protonated form of procaine, quinine and N-methylglucosamine, and the protonated forms of basic amino acids such as glycine, ornithine, histidine, phenylglycine, lysine and arginine. Furthermore, any zwitterionic form of the instant compounds formed by a carboxylic acid and an amino group are also contemplated.

The present invention also includes solvates of the compounds of the present invention and salts thereof. “Solvate” means a physical association of a compound of the invention with one or more solvent molecules. This physical association involves varying degrees of ionic and covalent bonding, including hydrogen bonding. In certain instances the solvate will be capable of isolation. “Solvate” encompasses both solution-phase and isolatable solvates. Non-limiting examples of suitable solvates include ethanolates, methanolates and the like. “Hydrate” is a solvate wherein the solvent molecule is water.

The present invention also includes polymorphs of the compounds of the present invention and salts thereof. The term “polymorph” refers to a particular crystalline state of a substance, which can be characterized by particular physical properties such as X-ray diffraction, IR spectra, melting point, and the like.

Scheme 1 generally represents various embodiments of the Aloe-emodin derivatives of the present invention. Such derivatives may be prepared in accordance with the processes as described herein. Some currently preferred embodiments are described hereinbelow.

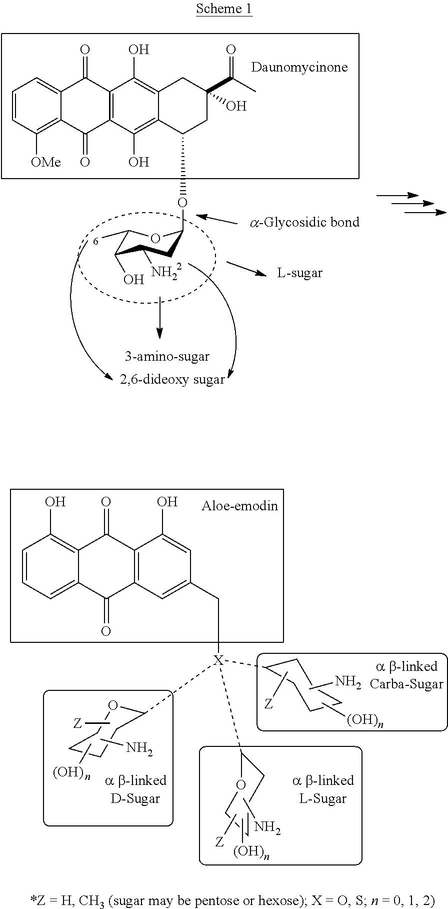

### Aloe-Emodin Based Anthraquinone Scaffolds:

Scheme 2 lists several scaffolds of Aloe-emodin that are suitable for use in the context of the present invention. Any combination of the R¹, R² and R³ substituents are contemplated within the broad scope of the present invention.

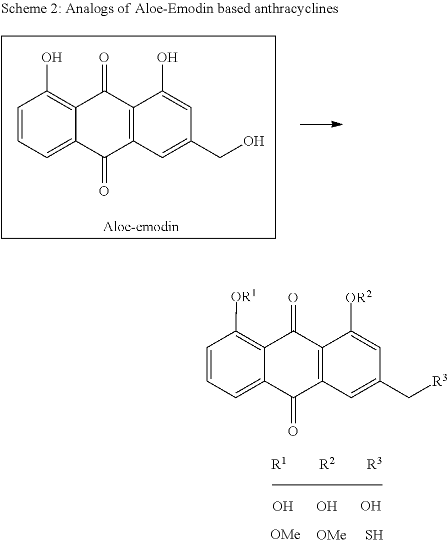

To date, anthracyclines cannot be administered orally due to the cleavage of the sugar side chains in the acidic environment of the stomach. To address this issue, benzylic thiol analogs of Aloe-emodin were prepared by replacing the alcohol of R₃=OH to R³=SH (Scheme 2). Furthermore, to improve the hydrophobic based target DNA stacking interactions of the anthraquinone scaffold, methylated analogs of Aloe-emodin may be used. Aloe-emodin and/or its thio analogs can be methylated on either one or both phenolic alcohols (R¹=methyl, R²=methyl or both R¹ and R²=methyl, Scheme 2).

### Sugar and Carba-Sugar Scaffolds to be Attached to the Aloe-Emodin Scaffold:

The present invention contemplates a variety of sugar and carba-sugars derivatives to be attached to the Aloe-emodin scaffold. Currently preferred sugars to be used in the present invention are 2-deoxy pyranose form of common aldopentoses and aldohexoses (Scheme 3).

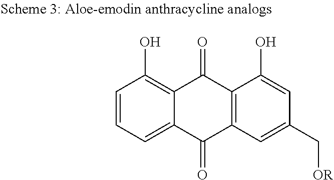

### R=

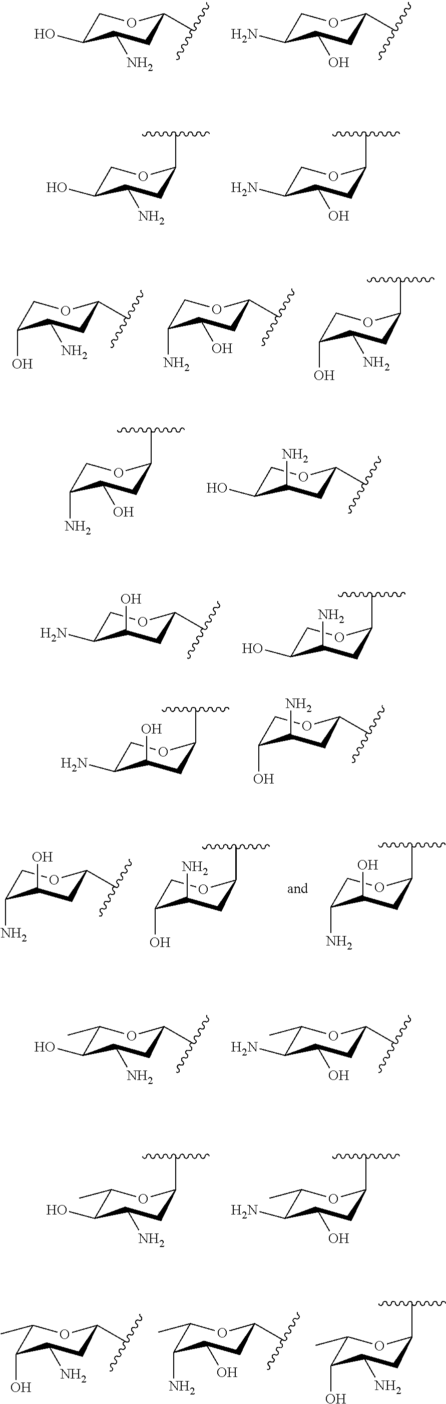

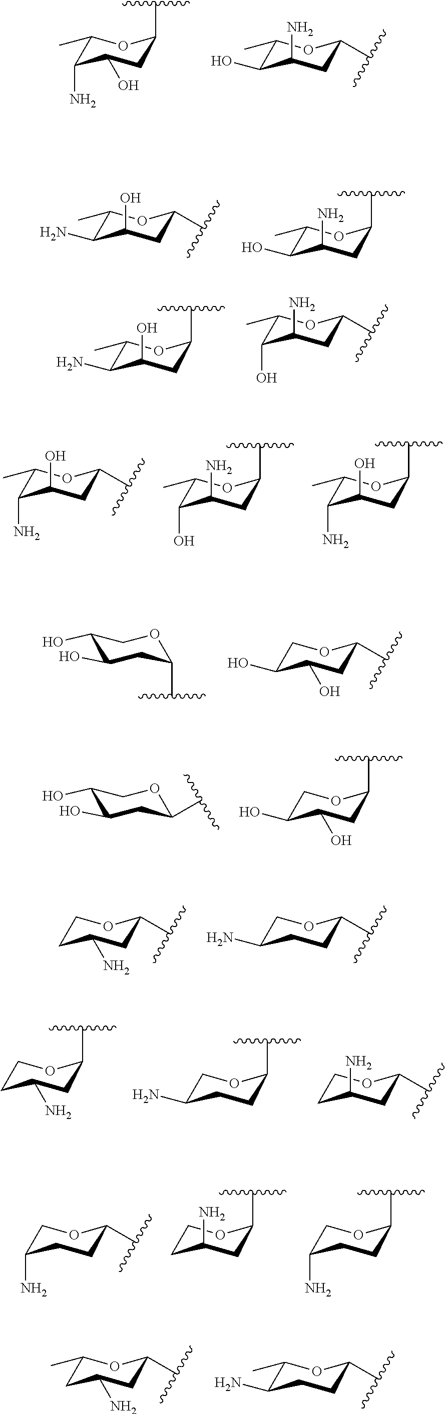

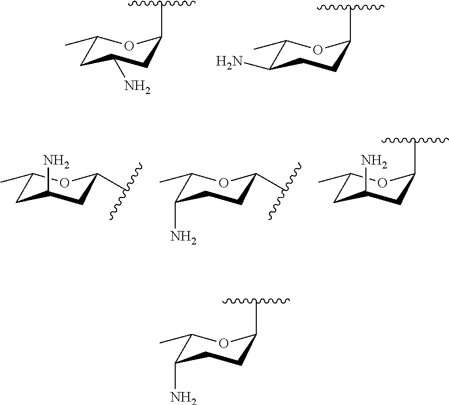

In some embodiments, the sugar is an aldopentose derived from 2-deoxy-D or L-ribose. In other embodiments, the sugar is an aldohexose derived from 2-deoxy-D or L-rhamnose. However, it should be apparent to a person of skill in the art that Aloe-emodin derivatives containing other sugars, especially in the pyranose forms of any aldopentoses and aldohexoses, are also encompassed by the present invention.

In other embodiments, the sugar in the compound of formula (I) (i.e., R³) is an amino sugar, preferably in the form of an acetal or a thioacetal of an amino sugar). The amino sugar may be a 2-deoxy amino sugar, a 3-deoxy amino sugar, a 6-deoxy amino sugar, a 2,3-dideoxy amino sugar, a 2,6-dideoxy amino sugar, a 3,6-dideoxy amino sugar or a 2,3,6-trideoxy amino sugar. Each possibility represents a separate embodiment of the present invention. In one currently preferred embodiment, the amino group is at the C-3 position, thus representing a 3-amino sugar. The 3-amino sugar may be a 2-deoxy-3-amino sugar, a 3-deoxy-3-amino sugar, a 6-deoxy-3-amino sugar, a 2,3-dideoxy-3-amino sugar, a 2,6-dideoxy 3-amino sugar, a 3,6-dideoxy-3-amino sugar, or more preferably a 2,3,6-trideoxy-3-amino sugar. 4-deoxy amino sugars, including 2,4-deoxy, 3,4-deoxy, 4,6-deoxy, 2,3,4-trideoxy, 3,4,6-trideoxy, 2,4,6-trideoxy and 2,3,4,6-tetradeoxy amino sugars are also contemplated. Each possibility represents a separate embodiment of the present invention.

Preferably, the amino sugar is a 2-deoxypyranose form of an aldohexose. In other embodiments, however, the amino sugar is a 2-deoxy pyranose form of an aldopentose. Each possibility represents a separate embodiment of the present invention.

In some embodiments, the sugars are 2-deoxy sugars and contain an equatorial amine at position C-3 (compounds (i)-(iv) show several precursors of such sugars). Without wishing to be bound by any particular mechanism or theory, it is contemplated that these structural and functional features are significant for the target DNA minor groove binding. Two possible ring conformations may be prepared, so as to optimize the biological performance. In one embodiment, 2-deoxy-D and L-ribose can be used as starting materials. In some embodiments, the sugar scaffolds have no substituent at the C-5-position. In alternative embodiments, however, the sugars carry a methyl substitution at C-5, as in common sugars found on anthracyclines. A combination of ester protecting groups on the hydroxyls and azide protecting groups as amine precursors enable a mild single step procedure for the final synthetic step of protecting group removal. Several representative amino-sugar building blocks (compound 13-16) are set forth below:

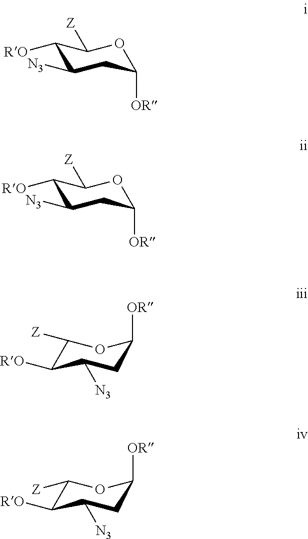

OR′=protected OH group; e.g., OBz, OAc

Z=H (pentose); CH₃ (hexose)

OR″—e.g., OAc,

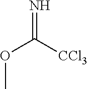

Similar considerations are applied to the preparation of carba-sugars. Several representative carba amino-sugar building blocks (compound v-viii) are set forth below:

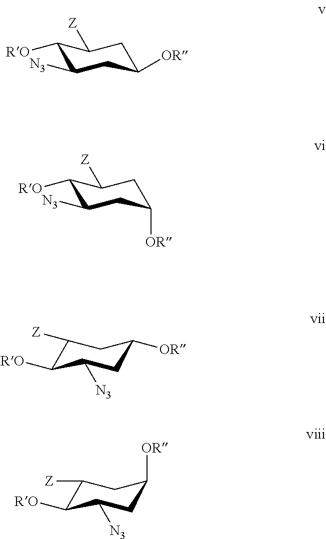

wherein Z, OR′ and OR″ are as defined above for compounds i-iv.

The compounds of formula (I) may be prepared by a process comprising the step of coupling a compound of formula (II)

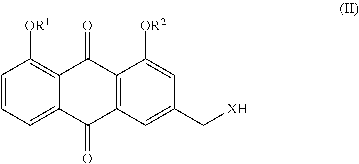

or an activated derivative thereof, wherein R¹, R² and X are as defined above for formula (I) optionally in the presence of a catalyst, with an amino sugar or amino carba-sugar derivative of formula R³—Y wherein Y is a leaving group, so as to generate a compound of formula (I).

In one particular embodiment, R³ is an amino sugar, and the process comprises the following steps: (i) coupling a compound of formula (II) or an activated derivative thereof, optionally in the presence of a catalyst, with an amino sugar derivative represented by the structure of formula (III):

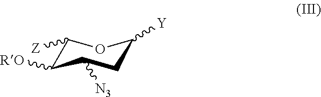

wherein Y is a leaving group as defined herein, R′ is a hydroxyl protecting group, Z is H or CH₃, wherein the substituents Z, OR′, N₃ and Y can each independently be in the equatorial or axial position, so as to generate a compound of formula (IV):

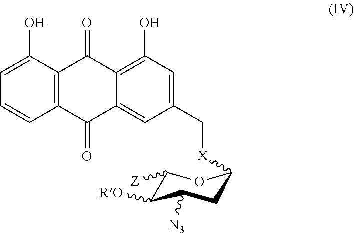

(ii) removing the hydroxy protecting group R′ to generate a free hydroxyl; and (iii) converting the azide group (N₃) to an amine (NH₂); wherein steps (ii) and (iii) can be conducted in any order.

When Z=H, the amino sugar is a pentose, and when Z=CH₃, the amino sugar is a hexose. Currently preferred sugars are hexoses. Each possibility represents a separate embodiment of the present invention.

As used herein, the term “OH protecting group” or “hydroxy protecting group” refers to a readily cleavable groups) bonded to hydroxyl groups. The nature of the hydroxy-protecting groups is not critical so long as the derivatized hydroxyl group is stable. An example of a hydroxy protecting group is an acyl group (COR wherein R=alkyl, aryl, etc.). A currently preferred acyl group is an acetyl group (i.e., OR′=acetate, OAc). Another example of a hydroxy protecting group is a silyl group, which can be substituted with alkyl (trialkylsilyl), with an aryl (triarylsilyl) or a combination thereof (e.g., dialkylphenylsilyl). A preferred example of a silyl protecting group is trimethylsilyl (TMS) or di-t-butyldimethyl silyl (TBDMS). Other examples of hydroxy protecting groups include, for example, C₁-C₄ alkyl, —CO—(C₁-C₆ alkyl), —SO₂—(C₁-C₆ alkyl), —SO₂-aryl, —CO—Ar in which Ar is an aryl group as defined above, and —CO—(C₁-C₆ alkyl)Ar (e.g., a carboxybenzyl (Bz) group). Other examples of hydroxy-protecting groups are described by C. B. Reese and E. Haslam, “Protective Groups in Organic Chemistry,” J. G. W. McOmie, Ed., Plenum Press, New York, N.Y., 1973, Chapters 3 and 4, respectively, and T. W. Greene and P. G. M. Wuts, “Protective Groups in OrganicSynthesis,”2nd ed., John Wiley and Sons, New York, N.Y., 1991, Chapters 2 and 3, each of which is incorporated herein by reference.

As used herein, the term “leaving group” (i.e, group Y) refers to any labile group. An example of a leaving group is a moiety of formula OR″ wherein R″ can be any hydroxy protecting group as defined above. Some currently preferred embodiments are Y═OR″=OAc or —O—(C═N)—CCl₃. Other suitable leaving groups are, for example, halogen, e.g. chlorine, bromine or iodine, or an organosulfonyloxy radical (OSO₂R′), for example, mesyloxy, tosyloxy, trifloxy and the like. Each possibility represents a separate embodiment of the present invention.

An activated derivative of a group XH can be for example —OR —SR wherein R is alkyl, aryl, acyl, etc., as known to a person of skill in the art.

### Therapeutic Uses

The present invention relates to a method for treating cancer in a subject in need thereof, comprising administering to the subject a compound of formula (I), or salts, solvates, polymorphs, optical isomers, geometrical isomers, enantiomers, diastereomers, and mixtures thereof.

In another embodiment, the present invention relates to the use of a compound of formula (I) for the preparation of a medicament for the treatment of cancer.

In one embodiment, the compound of formula (I) is represented by the structure of formula (II). In another embodiment, the compound of formula (I) is represented by the structure of formula (12). In another embodiment, the compound of formula (I) is represented by the structure of formula (13). In another embodiment, the compound of formula (I) is represented by the structure of formula (14). In another embodiment, the compound of formula (15) is represented by the structure of formula (16).

The term “cancer” in the context of the present invention includes all types of neoplasm whether in the form of solid or non-solid tumors, from all origins, and includes both malignant and premalignant conditions as well as their metastases. The combinations of the present invention are active against a wide range of cancers including, but are not limited to, leukemia, sarcoma, melanoma, carcinoma, T-cell leukemia, adenocarcinoma, fibrosarcoma, lymphoproliferative disorders, breast cancer, ovarian cancer, prostate cancer, cervical cancer, endometrial cancer, bone cancer, liver cancer, stomach cancer, colon cancer, pancreatic cancer, cancer of the thyroid, head and neck cancer, cancer of the central nervous system, cancer of the peripheral nervous system, skin cancer and kidney cancer, as well as metastases of all the above. Other types of cancer include, but are not limited to hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, squamous cell carcinoma, basal cell carcinoma, adenocarcinoma (well differentiated, moderately differentiated, poorly differentiated or undifferentiated), renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma and non-Hodgkin's lymphoma, as well as metastases of all of the above.

In some embodiments, the cancer to be treated is characterized by resistance to anthracycline chemotherapeutic agents. As mentioned above, it has unexpectedly been discovered that these AEG derivatives exhibit potent activity against various tumors that have shown resistance to conventional anthracycline chemotherapeutic agents such as doxorubicin. As such, the compounds of the invention offer significant advantages over conventional anthracycline-based chemotherapeutic agents.

Examples of anthracyline-resistant cancers that are treatable with the compounds of the invention include, but are not limited to lymphoproliferative disorders, breast cancer, ovarian cancer, prostate cancer, colon cancer, pancreatic cancer, sarcomas, fibrosarcoma, melanoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma and non-Hodgkin's lymphoma, as well as metastases of all of the above.

In addition, essentially any cancer that has shown some type of resistance to anthracycline agents is treatable with the AEG derivatives of the present invention. Additional examples of such cancers are disclosed, e.g., in Broxterman et al, the contents of which are incorporated by reference herein. ^{[15]}

In one embodiment, the subject is a mammal, preferably a human. However, the present invention also contemplates using the compounds of the present invention for non-mammal humans, e.g., in veterinary medicine.

It is to be understood that whenever the terms “treating or inhibiting cancer” or “treating or inhibiting a malignant (cancer) cell proliferation” are used herein in the description and in the claims, they are intended to encompass tumor formation, primary tumors, tumor progression or tumor metastasis.

The term “inhibition of proliferation” in relation to cancer cells, in the context of the present invention refers to a decrease in at least one of the following: number of cells (due to cell death which may be necrotic, apoptotic or any other type of cell death or combinations thereof) as compared to control; decrease in growth rates of cells, i.e. the total number of cells may increase but at a lower level or at a lower rate than the increase in control; decrease in the invasiveness of cells (as determined for example by soft agar assay) as compared to control even if their total number has not changed; progression from a less differentiated cell type to a more differentiated cell type; a deceleration in the neoplastic transformation; or alternatively the slowing of the progression of the cancer cells from one stage to the next.

The term “treatment of cancer” in the context of the present invention includes at least one of the following: a decrease in the rate of growth of the cancer (i.e. the cancer still grows but at a slower rate); cessation of growth of the cancerous growth, i.e., stasis of the tumor growth, and, in preferred cases, the tumor diminishes or is reduced in size. The term also includes reduction in the number of metastases, reduction in the number of new metastases formed, slowing of the progression of cancer from one stage to the other and a decrease in the angiogenesis induced by the cancer. In most preferred cases, the tumor is totally eliminated. Additionally included in this term is lengthening of the survival period of the subject undergoing treatment, lengthening the time of diseases progression, tumor regression, and the like. This term also encompasses prevention for prophylactic situations or for those individuals who are susceptible to contracting a tumor. The administration of the compounds of the present invention will reduce the likelihood of the individual contracting the disease. In preferred situations, the individual to whom the compound is administered does not contract the disease.

As used herein, the term “administering” refers to bringing in contact with a compound of the present invention. Administration can be accomplished to cells or tissue cultures, or to living organisms, for example humans. In one embodiment, the present invention encompasses administering the compounds of the present invention to a human subject.

A “therapeutic” treatment is a treatment administered to a subject who exhibits signs of pathology for the purpose of diminishing or eliminating those signs. A “therapeutically effective amount” is that amount of compound which is sufficient to provide a beneficial effect to the subject to which the compound is administered. A “synergistic therapeutically effective amount” means that the combination treatment regimen produces a significantly better anticancer result (e.g., cell growth arrest, apoptosis, induction of differentiation, cell death) than the additive effects of each constituent when it is administered alone at a therapeutic dose. Standard statistical analysis can be employed to determine when the results are significantly better. For example, a Mann-Whitney Test or some other generally accepted statistical analysis can be employed.

### Pharmaceutical Compositions

Although the compounds of the present invention can be administered alone, it is contemplated that such compounds will be administered in pharmaceutical compositions further containing at least one pharmaceutically acceptable carrier or excipient. Where combination treatments are used, each of the components can be administered in a separate pharmaceutical composition, or the combination can be administered in one pharmaceutical composition.

Thus, in one embodiment, the present invention also contemplates pharmaceutical compositions that comprise a compound of formula (I), and a pharmaceutically acceptable excipient. In one embodiment, the compound of formula (I) is represented by the structure of formula (II). In another embodiment, the compound of formula (I) is represented by the structure of formula (12). In another embodiment, the compound of formula (I) is represented by the structure of formula (13). In another embodiment, the compound of formula (I) is represented by the structure of formula (14). In another embodiment, the compound of formula (I) is represented by the structure of formula (15). In another embodiment, the compound of formula (I) is represented by the structure of formula (16). Each possibility represents a separate embodiment of the present invention.

The pharmaceutical compositions of the present invention can be formulated for administration by a variety of routes including oral, rectal, transdermal, parenteral (subcutaneous, intraperitoneal, intravenous, intra-arterial, transdermal and intramuscular), topical, intranasal, or via a suppository. Such compositions are prepared in a manner well known in the pharmaceutical art and comprise as an active ingredient at least one compound of the present invention as described hereinabove, and a pharmaceutically acceptable excipient or a carrier. The term “pharmaceutically acceptable” means approved by a regulatory agency of the Federal or a state government or listed in the U.S. Pharmacopeia or other generally recognized pharmacopeia for use in animals and, more particularly, in humans.

During the preparation of the pharmaceutical compositions according to the present invention the active ingredient is usually mixed with a carrier or excipient, which may be a solid, semi-solid, or liquid material. The compositions can be in the form of tablets, pills, capsules, pellets, granules, powders, lozenges, sachets, cachets, elixirs, suspensions, dispersions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

The carriers may be any of those conventionally used and are limited only by chemical-physical considerations, such as solubility and lack of reactivity with the compound of the invention, and by the route of administration. The choice of carrier will be determined by the particular method used to administer the pharmaceutical composition. Some examples of suitable carriers include lactose, glucose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water and methylcellulose. The formulations can additionally include lubricating agents such as talc, magnesium stearate, and mineral oil; wetting agents, surfactants, emulsifying and suspending agents; preserving agents such as methyl- and propylhydroxybenzoates; sweetening agents; flavoring agents, colorants, buffering agents (e.g., acetates, citrates or phosphates), disintegrating agents, moistening agents, antibacterial agents, antioxidants (e.g., ascorbic acid or sodium bisulfite), chelating agents (e.g., ethylenediaminetetraacetic acid), and agents for the adjustment of tonicity such as sodium chloride. Other pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions.

In one embodiment, in the pharmaceutical composition the active ingredient is dissolved in any acceptable lipid carrier (e.g., fatty acids, oils to form, for example, a micelle or a liposome).

In some embodiments, nanocarriers are used to effectuate intracellular uptake or transcellular transport of the compounds of the invention. Nanocarriers are miniature devices or particles that can readily interact with biomolecules on cell surfaces and within cells. Pharmaceutical nanocarriers such as viral vectors, polymeric nanoparticles, and liposomes are advantageous for delivering pharmaceutically active agents more selectively to target cells. Nanocarriers also effectively enhance the delivery of poorly-soluble therapeutics and control the release rate of encapsulated compounds. Many nanocarriers are natural or synthetic polymers that have defined physical and chemical characteristics. As a consequence, a person skilled in the art can engineer desired properties, such as target selectivity, biodegradability, biocompatibility, and responsiveness to environmental factors (e.g., pH or temperature changes), into nanocarriers to improve performance. Any nanocarriers can be used in the context of the present invention.

For preparing solid compositions such as tablets, the principal active ingredient(s) is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, from about 0.1 mg to about 2000 mg, from about 0.1 mg to about 500 mg, from about 1 mg to about 100 mg, from about 100 mg to about 250 mg, etc. of the active ingredient(s) of the present invention.

Any method can be used to prepare the pharmaceutical compositions. Solid dosage forms can be prepared by wet granulation, dry granulation, direct compression and the like. The solid dosage forms of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compositions of the present invention may be incorporated, for administration orally or by injection, include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insulation include solutions and suspensions in pharmaceutically acceptable aqueous or organic solvents, or mixtures thereof, and powders. The liquid or solid compositions may contain suitable pharmaceutically acceptable excipients as described above. Preferably the compositions are administered by the oral or nasal respiratory route for local or systemic effect. Compositions in preferably pharmaceutically acceptable solvents may be nebulized by use of inert gases. Nebulized solutions may be breathed directly from the nebulizing device or the nebulizing device may be attached to a face masks tent, or intermittent positive pressure breathing machine. Solution; suspension, or powder compositions may be administered, preferably orally or nasally, from devices that deliver the formulation in an appropriate manner.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art.

In yet another embodiment, the composition is prepared for topical administration, e.g. as an ointment, a gel a drop or a cream. For topical administration to body surfaces using, for example, creams, gels, drops, ointments and the like, the compounds of the present invention can be prepared and applied in a physiologically acceptable diluent with or without a pharmaceutical carrier. The present invention may be used topically or transdermally to treat cancer, for example, melanoma. Adjuvants for topical or gel base forms may include, for example, sodium carboxymethylcellulose, polyacrylates, polyoxyethylene-polyoxypropylene-block polymers, polyethylene glycol and wood wax alcohols.

Alternative formulations include nasal sprays, liposomal formulations, slow-release formulations, pumps delivering the drugs into the body (including mechanical or osmotic pumps) controlled-release formulations and the like, as are known in the art.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In preparing a formulation, it may be necessary to mill the active ingredient to provide the appropriate particle size prior to combining with the other ingredients. If the active compound is substantially insoluble, it ordinarily is milled to a particle size of less than 200 mesh. If the active ingredient is substantially water soluble, the particle size is normally adjusted by milling to provide a substantially uniform distribution in the formulation, e.g. about 40 mesh.

It may be desirable to administer the pharmaceutical composition of the invention locally to the area in need of treatment; this may be achieved by, for example, and not by way of limitation, local infusion during surgery, infusion to the liver via feeding blood vessels with or without surgery, topical application, e.g., in conjunction with a wound dressing after surgery, by injection, by means of a catheter, by means of a suppository, or by means of an implant, said implant being of a porous, non-porous, or gelatinous material. According to some preferred embodiments, administration can be by direct injection e.g., via a syringe, at the site of a tumor or neoplastic or pre-neoplastic tissue.

The compounds may also be administered by any convenient route, for example by infusion or bolus injection, by absorption through epithelial linings (e.g., oral mucosa, rectal and intestinal mucosa, etc.), and may be administered together with other therapeutically active agents. It is preferred that administration is localized, but it may be systemic. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer, and formulation with an aerosolizing agent.

A compound of the present invention can be delivered in an immediate release or in a controlled release system. In one embodiment, an infusion pump may be used to administer a compound of the invention, such as one that is used for delivering chemotherapy to specific organs or tumors (see Buchwald et al., 1980, Surgery 88: 507; Saudek et al., 1989, N. Engl. J. Med. 321: 574). In a preferred form, a compound of the invention is administered in combination with a biodegradable, biocompatible polymeric implant, which releases the compound over a controlled period of time at a selected site. Examples of preferred polymeric materials include polyanhydrides, polyorthoesters, polyglycolic acid, polylactic acid, polyethylene vinyl acetate, copolymers and blends thereof (See, Medical applications of controlled release, Langer and Wise (eds.), 1974, CRC Pres., Boca Raton, Fla.). In yet another embodiment, a controlled release system can be placed in proximity of the therapeutic target, thus requiring only a fraction of the systemic dose.

Furthermore, at times, the pharmaceutical compositions may be formulated for parenteral administration (subcutaneous, intravenous, intraarterial, transdermal, intraperitoneal or intramuscular injection) and may include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. Oils such as petroleum, animal, vegetable, or synthetic oils and soaps such as fatty alkali metal, ammonium, and triethanolamine salts, and suitable detergents may also be used for parenteral administration. The above formulations may also be used for direct intra-tumoral injection. Further, in order to minimize or eliminate irritation at the site of injection, the compositions may contain one or more nonionic surfactants. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described and known in the art.

Alternatively, the compounds of the present invention can be used in hemodialysis such as leukophoresis and other related methods, e.g., blood is drawn from the patient by a variety of methods such as dialysis through a column/hollow fiber membrane, cartridge etc, is treated with the compounds of the invention Ex-vivo, and returned to the patient following treatment. Such treatment methods are well known and described in the art. See, e.g., Kolho et al. (J. Med. Virol. 1993, 40(4): 318-21); Ting et al. (Transplantation, 1978, 25(1): 31-3); the contents of which are hereby incorporated by reference in their entirety.

### Doses and Dosing Schedules

The amount of a compound of the invention that will be effective in the treatment of a particular disorder or condition, including cancer, will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. A preferred dosage will be within the range of 0.01-1000 mg/kg of body weight, more preferably, 0.1 mg/kg to 100 mg/kg and even more preferably 1 mg/kg to 10 mg/kg. Effective doses may be extrapolated from dose-response curves derived from in vitro or animal model test bioassays or systems. When a synergistic effect is observed, the overall dose of each of the components may be lower, thus the side effects experienced by the subject may be significantly lower, while a sufficient chemotherapeutic effect is nevertheless achieved.

The administration schedule will depend on several factors such as the cancer being treated, the severity and progression, the patient population, age, weight etc. For example, the compositions of the invention can be taken once-daily, twice-daily, thrice daily, once-weekly or once-monthly. In addition, the administration can be continuous, i.e., every day, or intermittently. The terms “intermittent” or “intermittently” as used herein means stopping and starting at either regular or irregular intervals. For example, intermittent administration can be administration one to six days per week or it may mean administration in cycles (e.g. daily administration for two to eight consecutive weeks, then a rest period with no administration for up to one week) or it may mean administration on alternate days. The different components of the combination can, independently of the other, follow different dosing schedules.

### Combination Therapy

The compounds of the present invention may be used alone, or they may be used in combination with other conventional chemotherapeutic agents. Suitable chemotherapeutic agents for use in the combinations of the present invention include, but are not limited to, alkylating agents, antibiotic agents, antimetabolic agents, hormonal agents, plant-derived agents, anti-angiogenic agents, differentiation inducing agents, cell growth arrest inducing agents, apoptosis inducing agents, cytotoxic agents, agents affecting cell bioenergetics, biologic agents, e.g., monoclonal antibodies, kinase inhibitors and inhibitors of growth factors and their receptors, gene therapy agents, cell therapy, e.g., stem cells, or any combination thereof.

Alkylating agents are drugs which impair cell function by forming covalent bonds with amino, carboxyl, suflhydryl and phosphate groups in biologically important molecules. The most important sites of alkylation are DNA, RNA and proteins. Alkylating agents depend on cell proliferation for activity but are not cell-cycle-phase-specific. Alkylating agents suitable for use in the present invention include, but are not limited to, bischloroethylamines (nitrogen mustards, e.g. chlorambucil, cyclophosphamide, ifosfamide, mechlorethamine, melphalan, uracil mustard), aziridines (e.g. thiotepa), alkyl alkone sulfonates (e.g. busulfan), nitroso-ureas (e.g. BCNU, carmustine, lomustine, streptozocin), nonclassic alkylating agents (e.g., altretamine, dacarbazine, and procarbazine), and platinum compounds (e.g., carboplastin and cisplatin).

Antitumor antibiotics like adriamycin intercalate DNA at guanine-cytosine and guanine-thymine sequences, resulting in spontaneous oxidation and formation of free oxygen radicals that cause strand breakage (7). Other antibiotic agents suitable for use in the present invention include, but are not limited to, anthracyclines (e.g. doxorubicin, daunorubicin, epirubicin, idarubicin and anthracenedione), mitomycin C, bleomycin, dactinomycin, and plicatomycin.

Antimetabolic agents suitable for use in the present invention, include but are not limited to, floxuridine, fluorouracil, methotrexate, leucovorin, hydroxyurea, thioguanine, mercaptopurine, cytarabine, pentostatin, fludarabine phosphate, cladribine, asparaginase, and gemcitabine.

Hormonal agents suitable for use in the present invention, include but are not limited to, an estrogen, a progestogen, an antiesterogen, an androgen, an antiandrogen, an LHRH analogue, an aromatase inhibitor, diethylstibestrol, tamoxifen, toremifene, fluoxymesterol, raloxifene, bicalutamide, nilutamide, flutamide, aminoglutethimide, tetrazole, ketoconazole, goserelin acetate, leuprolide, megestrol acetate, and mifepristone.

Plant derived agents include taxanes, which are semisynthetic derivatives of extracted precursors from the needles of yew plants. These drugs have a novel 14-member ring, the taxane. Unlike the vinca alkaloids, which cause microtubular disassembly, the taxanes (e.g., taxol) promote microtubular assembly and stability, therefore blocking the cell cycle in mitosis (7). Other plant derived agents include, but are not limited to, vincristine, vinblastine, vindesine, vinzolidine, vinorelbine, etoposide, teniposide, and docetaxel.

Biologic agents suitable for use in the present invention include, but are not limited to immuno-modulating proteins, monoclonal antibodies against tumor antigens, tumor suppressor genes, kinase inhibitors, PARP inhibitors, mTOR inhibitors, AKT inhibitors, and inhibitors of growth factors and their receptors and cancer vaccines. For example, the immuno-modulating protein can be interleukin 2, interleukin 4, interleukin 12, interferon E1 interferon D, interferon alpha, erythropoietin, granulocyte-CSF, granulocyte, macrophage-CSF, bacillus Calmette-Guerin, levamisole, or octreotide. Furthermore, the tumor suppressor gene can be DPC-4, NF-1, NF-2, RB, p53, WT1, BRCA, or BRCA2. Combinations with stem cell therapy are also contemplated.

Agents affecting cell bioenergetics affect, e.g., cellular ATP levels and/or molecules/activities regulating these levels,

Recent developments have introduced, in addition to the traditional cytotoxic and hormonal therapies, additional therapies for the treatment of cancer. For example, many forms of gene therapy are undergoing preclinical or clinical trials. In addition, approaches are currently under development that are based on the inhibition of tumor vascularization (angiogenesis). The aim of this concept is to cut off the tumor from nutrition and oxygen supply provided by a newly built tumor vascular system. In addition, cancer therapy is also being attempted by the induction of terminal differentiation of the neoplastic cells. Suitable differentiation agents include hydroxamic acids, derivatives of vitamin D and retinoic acid, steroid hormones, growth factors, tumor promoters, and inhibitors of DNA or RNA synthesis. Also, histone deacetylase (HDAC) inhibitors are suitable chemotherapeutic agent to be used in the present invention.

### EXAMPLES

The following are non-limiting examples for the preparation of AE-based compounds attached to amino sugars and amino carba-sugars. It is apparent to a person of skill in the art that the present invention is not limited to the currently recited synthetic schemes and that variations in reaction conditions and reagents are possible and are encompassed by the broad scope of the present invention.

### Example 1

### General Methods, Cell Strains, Plasmids, Materials, and Instrumentation

### A. General Techniques

NMR spectra were recorded on Bruker instruments: Avance™ 400 (400 MHz for ¹H, 100.6 MHz for ¹³C) or Avance™ 500 (500 MHz for ¹H, 125.7 MHz for ¹³C). Chemical shifts are reported in unit parts per million (ppm). ¹H NMR spectra were calibrated as follows: CD₃OD (3.34 ppm). ¹³C NMR spectra were calibrated as follows: CD₃OD (49.86 ppm). Multiplicities are reported by using the following abbreviations: s=singlet, d=doublet, t=triplet, q=quartet, m=multiplet, dd=double doublet, ddd=double double doublet, dq=double quartet. Coupling constant (J) are given in Hertz. Unless otherwise mentioned, all reactions were conducted under argon atmosphere using anhydrous solvents. Reactions were monitored by electron spray ionization (ESI) mass spectrometry and recorded on a Waters 3100 mass detector. High resolution mass spectra were measured on a Waters Synapt instrument. AEGs were purified on an ECOM preparative HPLC system using a Phenomenex Luna axia 5 μm C-18 column (250 mm×21.20 mm). Size exclusion chromatography was performed on an LH-20 column (70 cm×1.4 cm).

All reagents were used without further purification and were purchased from Sigma Aldrich, Alfa aeser, and Carbosynth. Aloe-emodin was purchased from Molekula. The cell lines used in this study were as follows: acute lymphoblastic leukemia (Molt-4), ovarian carcinoma (OVCAR3, NAR), breast adenocarcinomas (MCF7), B16 (murine melanoma), HCT 116 (human colon adenocarcinoma), and MCA 105 (murine fibrosarcoma), that were purchased from ATCC (Manassas, Va.). All cell lines except Molt-4 were grown in Dulbecco's modified Eagle's medium supplemented with 10% FBS, 1 mM L-glutamine, 100 U/ml penicillin, 100 μg/ml streptomycin. Molt-4 cells were grown in RPMI-1640 with the same supplements. Cell culture supplies was purchased from Biological Industries, Beit-Haemek, Israel.

PBR322 plasmid was purchased from Fermentas. DNA gels were run on an Bio-Rad Laboratories Ltd. instrument. DiIC₁₈(5)-DS plasma membrane stain was purchased from Invitrogen. Imaging was performed using an Andor Revolution Imaging System equipped with a Yokogawa CSU-X1 Spinning Disk Unit and an iXon 897 back-illuminated EMCCD camera, and mounted on a custom made Olympus IX-Upright microscope.

### B. Cytotoxicity IC₅₀ Experiments Protocol

Cells (5×10³/well) were plated into 96-well microtiter plates and were allowed to adhere (except for the leukemic cells) before treatment. After 24 hours, the plates were added with 5 μL of AEGs or AE or DOX solutions at different concentrations. The cells were incubated for 24 h. Cell viability was determined using an XTT kit protocol (Biological Industries). Optical density (OD) was read at 490 nm with a VERSAmax microplate ELISA reader (Molecular Devices, Sunnyvale, Calif., USA). IC₅₀ values were determined as the concentrations in which the OD value of the tested compound reached 50% of the OD value of the control containing un-treated cells. All experiments were performed in triplicates and repeated three times. All cell lines were grown in Dulbecco's modified Eagle's medium (or RPMI-1640 for Molt-4 cells) supplemented with 10% FBS, 1 mM L-glutamine, 100 U/ml penicillin, 100 μg/ml streptomycin. Cells were maintained in a humidified chamber of 95% air 5% CO₂ at 37° C.

### C. Supercoiled Plasmid DNA Unwinding Gel Experiments Protocol

Eppendorf tubes containing 3 μL supercoiled plasmid PBR322 (0.167 μg/ml), 14 μL tris-HCl-di-natrium-EDTA (TE X1) and 5 μL, AE, or one of the AEGs 1-4 or doxorubicin, at different concentrations were incubated for 15 min at 37° C. The samples were then added with 2 μl of loading buffer, and loaded on a 1% agarose gel in tris-HCl-boric-acid-di-natrium-EDTA (TBE X1). Samples were on the gel run for 15 min at 50 V and an additional 225 min at 70V. The gels were then immersed in a 0.5 mg/ml solution of ethidium bromide shaken for 20 min., washed with DDW for 5 mM. DNA was visualized by UV illumination.

### D. Light Microscopy Protocol

Cells (1×10⁵/well) were plated into 24-well microtiter plates and incubated and allowed to adhere for 24 hours before being treated with one of the AEGs or DOX or AE at a concentration of 20 μM maintained in a humidified chamber of 95% air 5% CO₂ at 37° C. for an additional 24 hours. Cells micrographs were obtained using a light microscope (Olympus IX50-S8F2 inverted phase microscope) at x400 magnification.

### E. Confocal Microscopy Protocol

Cells (1×10⁵/well) were plated into 24-well microtiter plates and incubated over coverslips for 24 hours. The cells were then added with one of the AEGs or DOX or AE at a total sample concentration of 5 μM. After 2 hours of incubation, the cells were washed three times, using PBS×1 (0.5 ml per well), and incubated with paraformaldehyde (3.7% in PBS 0.5 ml per well). After 15 min at ambient temperature, the cells were washed twice using PBS×1 0.5 ml per well. Cells were shaken at 50 rpm for 5 min after each wash. The plasma membrane of the fixated cells was stained by incubation of the samples with 300 μl of DiIC₁₈(5)-DS (4×10′ g/L) at 4° C. After 20 min, the cells were washed twice using PBS×1 0.5 ml per well. Cells were shaken at 50 rpm for 5 min after each wash. Finally, the samples were placed on a microscope slide and fixed using 10 μl of mounting. The slides were observed using an Andor Revolution Imaging System equipped with a Yokogawa CSU-X1 Spinning Disk Unit and an iXon 897 back-illuminated EMCCD camera, and mounted on a custom made Olympus IX-Upright microscope at Ex/Em=488/525 nm for DOX and AE glycoside I and Ex/Em=640/685 nm for DiIC₁₈ (5)-DS.

### F. Nomenclature

The following exemplifies the numbering system used to designate certain compounds of the present invention:

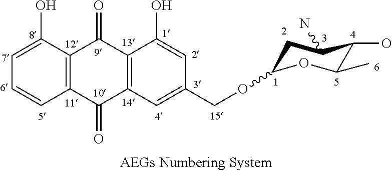

### Example 2

### Synthesis of Aloe-Emodin Derivatives Containing Amino Sugars and the Carba-Sugar Analogs

### A. Examples for the Synthesis of Pyranoside Pentoses

In one embodiment, the synthesis of pyranoside pentoses may advantageously be achieved using 2,4,6-trimethyl thiol as a thioglycoside. The bulky thiol reacts with, the per-acetylated pyranoside pentose and provides the beta-anomer preferentially (4a, 4b, Scheme 4). The thioglycoside 4b is then de-acetylated using, e.g., a mild variation of Zamplen de-acetylation to yield the diol 4c. Selective acetylation of the C-3-axial alcohol of 4c by the reaction of the diol with methyl orthoformate or an equivalent acetylating agent, preferably under acidic conditions followed by mild aqueous acidic hydrolysis of the orthoester yields compound 4d. The C-3 alcohol of 4d is converted to the corresponding mesylate leaving group 4e. Nucleophilic displacement of the mesylate of 4e with an azide affords compound 4f. Using the acetate salt as a nucleophile affords the aldopentose pyranoside thioglycoside 4 g.

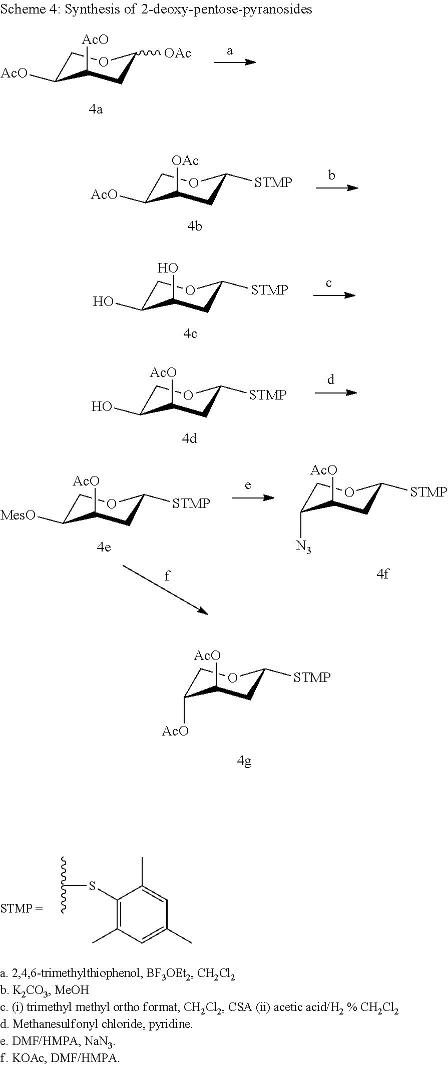

### B. Examples for Synthesis of Aloe-Emodin Anthracycline Analogs

For the coupling of the glycosyl donors any coupling protocols known in the art may be used. Two alternative and non-limiting coupling methods are described below.

In one embodiment, the glycosyl donors used in the present invention are activated using the AgPF₆ protocol.^{[24]} This protocol usually results in good yields and a relatively high a-selectivity. To obtain the β-anomers selectively, nitrile containing solvents such as acetonitrile or propionitrile at low temperatures may be used. The β-directing effect of the oxocarbenium stabilizing nitrilium ion leads to an increase in the formation β-configured products.^{[24]} Two variations may help enhance the β-directing effect: 1) the use of propionitrile as the reaction solvent which allows the reaction to take place at temperatures as low as −78° C.; and 2) the use of various ratios of dichloromethane to acetonitrile^{[25]}. In case a mixture of anomers is formed, flash chromatography may be used for the separation of the pure anomers as demonstrated in the experimental section. Activation of the glycosyl donor at low temperature may be achieved using the Schmidt trichloroacetimidate activation,^{[26]} For example, thioglycoside 5 is hydrolyzed using N-bromosuccinimide and the corresponding lactol is converted to the trichloroacetimidate glycosyl donor 5a by reacting the lactol with trichloacetonitrile under mild basic conditions (Scheme 5).^{[27]} Lewis acid catalyzed glycosidation of 5a and Aloe emodin at low temperature with propionitrile as the solvent affords the β-configured product. A single deprotection step, e.g., under basic conditions results in the hydrolysis of the benzoyl ester as well as with phosphine mediated conversion of the azide to the corresponding amine and yield the anthracycline analog 7.

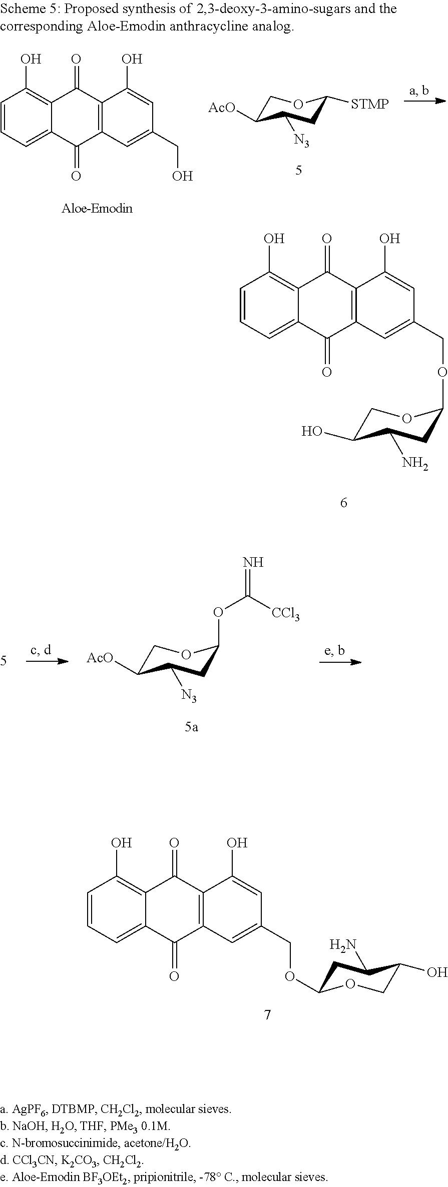

### C. Examples for Synthesis of Aloe-Emodin Carba-Sugar Analogs

Carba-sugars are widely used as sugar mimetics, and one of their main applications is for the inhibition of glycosidase and glycosyl transferase reactions.^{[28-30]} Thus, reductive as well as water mediated glycosidase enzymatic activity can be inhibited by carba-sugar anthracycline derivatives.

These building blocks can be synthesized by methods known in the art. For example, compound 8 can be synthesized from epoxide 8a,^{[31]} by protecting with a chloroacetyl group or other equivalent group (Scheme 6 compound 8b). Nucleophilic ring opening of 8b yields the product mixture of 8c and 8e. Benzoylation and selective removal of the chloroacetyl or equivalent group affords carba-sugar 8.

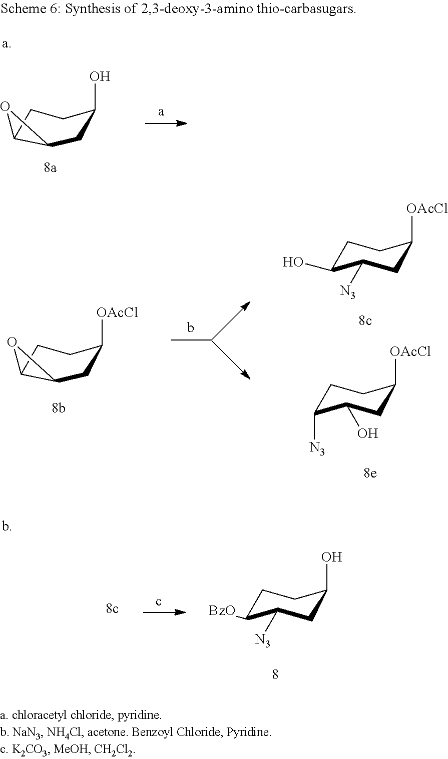

To attach the carba-sugars to the anthraquinone scaffold, a modified Bundle trichloroacetimidate based benzylation protocol may be used.^{[32]} The benzylic alcohol of Aloe-emodin is converted to the corresponding trichloroacetimidate Aloe-emodin derivative 8a. A Lewis acid catalyzed coupling of carba-sugar 8d and 9a results in the protected carba-sugar Aloe-emodin analog 9. A single deprotection step under the basic Staundinger reaction conditions results in the hydrolysis of the benzoyl ester as well as with phosphine mediated conversion of the azide to the corresponding amine and yield the carba-sugar analog of Aloe-emodin 9.

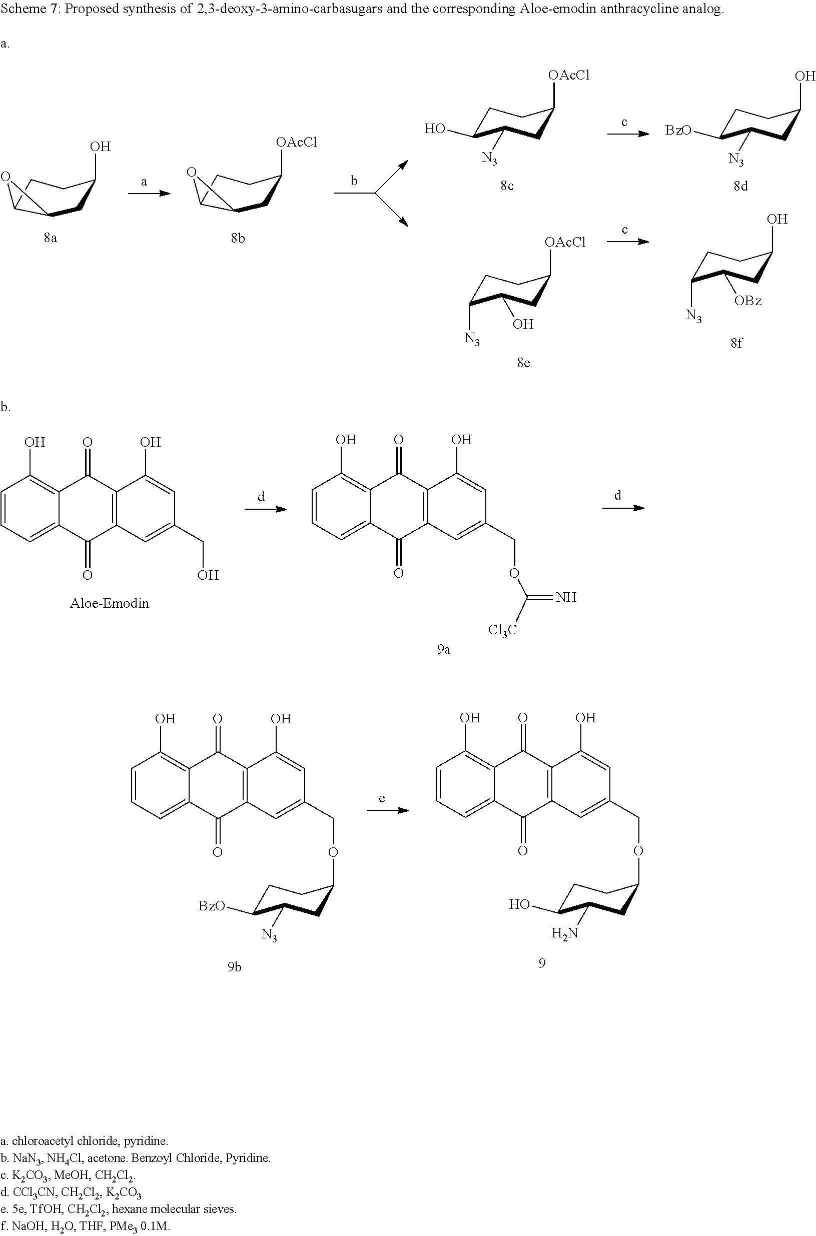

### Example 3

### Preparation of Pentose-Pyranoside-AEGs 11 and 12

The synthesis of the pentose-pyranoside-Aloe-emodin analogs 11(E1) and 12(E2) is demonstrated in scheme 8.

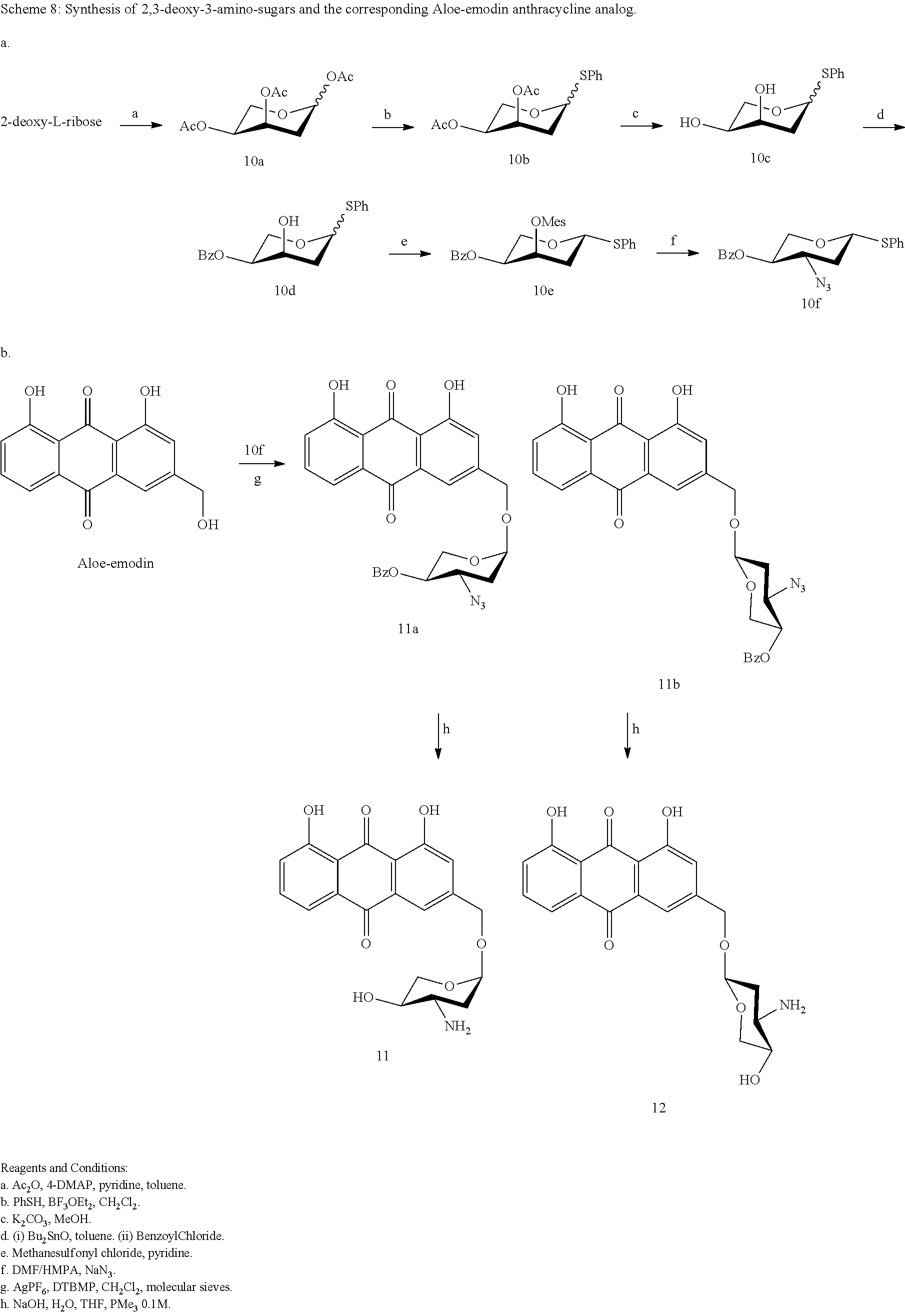

### 1,3,4-tri-O-acetyl-2-deoxy-L-ribopyranose 10a

2-deoxy-L-ribose (10 gr, 75 mmol) and 4-DMAP (200 mg, 2 mmol) were dissolved in dry toluene (50 ml). The mixture was stirred for 20 minutes at −40° C. and added with acetic anhydride (31 ml, 338 mmol) and pyridine (36 ml, 450 mmol). The reaction was kept in −40° C. for 2 h, and allowed to ambient temperature for 18 h. Reaction progress was monitored by TLC (75% petroleum ether, 25% ethyl acetate product R_{f}=0.33).

Upon termination, the reaction mixture was diluted with ethyl acetate (100 ml) and washed with HCl (0.2M), brine and NaHCO₃ sat. The combined organic phase was concentrated and crude mixture was purified by flash chromatography (silica, petroleum-ether/ethyl acetate). The product was obtained as a light yellow syrup mixture of α, β pyranosides and furanosides (16.1 gr, 62.0 mmol, 83%).

### Phenyl-3,4-di-O-acetyl-2-deoxy-thio-L-ribopyranose 10b

A solution of 10a (16.1 gr, 62.0 mmol) in DCM (70 ml) was added thiophenol (7 ml, 68 mmol) and cooled down to −50° C. After 10 minutes boron trifluoride diethyl etherate (400 μl, 3 μmol) was added. The reaction was kept in −50° C. for 4 h and then raised to 0° C. for the next 18 h. The reaction was monitored by TLC (75% petroleum ether, 25% ethyl acetate, product R_{f}=0.67). Upon termination, the reaction mixture was diluted with ethyl acetate (100 ml) and washed with sat. NaHCO₃ and brine. The organic phase was concentrated and crude mixture was purified by flash chromatography (silica, petroleum-ether/ethyl acetate) to yield a mixture of α, β pyranosides and furanosides, (9.66 gr, 31.1 mmol, 50%).

### Phenyl-2-deoxy-thio-L-ribopyranose 10c

Compound 10b, (9.66 gr, 31.1 mmol) was dissolved in methanol (100 ml) and added potassium carbonate (100 mg, 0.7 mmol). The reaction was stirred at ambient temperature for 2 h. The reaction was monitored by TLC (75% petroleum ether, 25% ethyl acetate, product R_{f}=0.15). Upon termination the reaction mixture was concentrated and crude was purified by flash chromatography (silica, petroleum-ether/ethyl acetate). The product was obtained as a mixture of α, β pyranosides and furanosides (6 gr, 26.5 mmol, 85%).

### Phenyl-4-O-benzoyl-2-deoxy-thio-L-ribopyranose 10d

A solution of 10c (2.51 gr, 11.1 mmol) in toluene (150 ml) was added dibutyltin oxide (4.1 g, 16.7 mmol). Using dean stark apparatus, the reaction was refluxed at 150° C. for 45 min. The reaction was then concentrated to 50 ml was then allowed to room temperature. Benzoyl chloride (1.41 mL, 12.21 mmol) was added and the solution was stirred for additional 3 hours at ambient temperature. Progress was monitored by TLC (75% petroleum ether, 25% ethyl acetate, product R_{f}=0.65). The mixture was then diluted with ethyl acetate (100 ml) and washed with HCl (0.2M), brine and NaHCO₃. Purification, by flash chromatography, (silica, petroleum-ether/ethyl acetate) yielded the β pyranose (940 mg, 286 mmol, 26%), and the α pyranose—as mixture of ¹C₄ and ⁴C₁, 3-O-benzoyl and 4-O-benzoyl (1.41 gr, 429 mmol, 40%). ¹H NMR (500 MHz, CDCl₃) for the β pyranose; δ 8.00 (m, 2H, benzoyl), 7.46-7.35 (m, 5H, aromatic), 7.27-7.17 (m, 5H, aromatic), 5.55 (t, J=4.2 Hz, 1H, H-1), 2.17 (m, 1H, H-4), 4.24 (m, 1H, H-3), 3.89 (d, J=4.5 Hz, 1H, H-5), 3.86 (d, J=4.5 Hz, 1H, H-5), 2.35 (m, 1H, H-2), 2.08 (m, 1H, H-2).

### Phenyl-4-O-benzoyl-3-O-methylsulfonyl-2-deoxy-thio-β-L-ribopyranose 10e

Compound 10d, (600 mg, 1.8 mmol) was dissolved in pyridine (10 mL) and the solution was cooled to 0° C. The mixture was then added methanesulfonyl chloride (281 μL, 3.6 mmol) and stirred at 0° C. for 1 h after which the temperature was allowed to room temperature. The reaction was monitored by TLC (75% petroleum ether, 25% ethyl acetate product R_{f}=0.65 similar to that of the starting material). Upon termination, the mixture was diluted with ethyl acetate (30 ml) and washed with HCl (0.2M), brine and NaHCO₃ sat. Flash chromatography (silica, petroleum-ether/ethyl acetate) afforded the product as light yellow syrup (465 mg, 1.04 mmol, 57%). ¹H NMR (500 MHz, CDCl3) 7.99 (m, 2H, benzoyl), 7.43-7.37 (m, 5H, aromatic), 7.29-7.14 (m, 5H, aromatic), 5.48 (dd, J₁=J₂=4.7 Hz, 1H, H-1), 5.32 (m, 1H, H-4), 5.21 (m, 1H, H-3), 4.28 (dd, J₁=2.7 Hz, J₂=12.5 Hz, 1H, H-5), 3.90 (dd, J₁=5.7 Hz, J₂=12.5 Hz, 1H, H-5), 2.94 (s, 3H, CH₃ of mesyl) 2.62 (m, 1H, H-2), 2.23 (m, 1H, H-2).

### Phenyl-4-O-benzoyl-3-deoxy-3-azido-2-deoxy-thio-fi-L-xylopyranose 10f

To compound 10e, (465 mg, 1.14 mmol) was added sodium azide (222 mg, 3.42 mmol), in HMPA (3 ml) and stirred for 4 h at 0° C. The reaction was monitored by TLC (75% petroleum ether, 25% ethyl acetate product R_{f}=0.75). Upon termination, the mixture reaction mixture was diluted with ethyl acetate (30 ml) and washed with brine. Purification by flash chromatography (silica-gel, petroleum-ether/ethyl acetate) afforded the product as a light yellow syrup (297 mg, 0.84 mmol, 73%). ¹H NMR (500 MHz, CDCl₃) δ 7.97 (m, 2H, benzoyl), 7.43-7.37 (m, 5H, aromatic), 7.27-7.14 (m, 5H, aromatic), 4.88 (ddd, J₁=4.5 Hz, J₂=8.5 Hz, J₃=13 Hz, 1H, H-4), 4.84 (dd, J₁=6.35 Hz, J₂=9.25 Hz, 1H, H-1), 4.28 (dd, J₁=4.5 Hz, J₂=11.4 Hz, 1H, H-5), 3.73 (m, 1H, H-3), 3.31 (dd, J₁=8 Hz, J₂=11.95 Hz, 1H, H-5), 2.34 (m, 1H, H-2), 1.78 (m, 1H, H-2). Compounds 11a and 11b: Compound 10f, (150 mg, 422 μmol) with Aloe emodin (137 mg, 506 μmol), and 2,6-di-tert-butyl-4-methylpyridine (346 mg, 1.67 mmol) were dissolved in dry dichloromethane (5 mL), under argon, and stirred for 40 min with freshly flame dried molecular sieves (200 mg). Silver hexafluorophosphate (533 mg, 2.54 mmol) was then added and the reaction was stirred at ambient temperature for 16 h. The reaction was monitored by TLC (90% acetone, 10% methanol) product Rf=0.21). Upon termination, the mixture was washed through a celite plug, concentrated and loaded on a silica-gel column. The crude was eluted with 90% acetone, 10% methanol and the products were obtain as a mixture of anomers 11a and 11b (80 mg, 156 μmol, 37%).

Compounds 11 (also referred to herein as “E-1”) and 12 (also referred to herein as “E-2”) mixture of 11a and 11b, (40 mg, 78 μmol) was dissolved in THF (800 μl), NaOH (0.1M, 200 μl), and methylamine solution 40% in water. The reaction was stirred for 3 h at 50° C. and then allowed to reach to room temperature. Trimethyl phosphine (1M in THF 310 μl, 310 μmol) was added and the reaction was stirred at ambient temperature for an additional 2 h. Monitoring was done by TLC (84% Chloroform, 15% methanol, 1% acetic acid, product R_{f}=0.2). Upon termination, the mixture was loaded on semipreparative HPLC to purify the anthracycline derivative. The purification was performed on a C18 Luna (250 mm×21.20 mm) column. The HPLC solvents were as follows: A, H2O (0.1% TFA); and B, MeCN (0.1% TFA). The elution gradient was: for 10 min 5% B, from 5% to 100% B over 40 min, 100% B over 5 min, and then from 100% to 5% B for 5 min. Product elution was monitored at 256 nm. The β anomer 12 was eluted first at 30.15 min, The α anomer 11 was eluted next at 30.33 min. ¹H NMR of 12 (500 MHz, MeOD) δ 7.70-7.65 (m, 3H), 7.23-7.21 (m, 1.5) 4.69 (dd, J₁=2.1 Hz, J₂=8.5 Hz, 1H, H-1), 4.47 (s, 1.5H), 3.93 (dd, J₁=4.8 Hz, J₂=11.7 Hz, 1H, H-5), 3.52 (ddd, J₁=4.95 Hz, J₂=9.1 Hz, J3=14 Hz, 1H, H-4), 3.24 (m, 1H, H-5), 3.10 (m, 1H, H-3), 2.25 (m, 1H, H-2), 1.64 (m, 1H, H-2); ¹³C NMR (500 MHz, MeOD) δ 192.1, 181.0, 148.1, 136.5, 127.6, 126.2, 123.7, 121.1, 120.4, 118.8, 117.2, 115.3, 107.2, 98.5, 77.5, 68.4, 66.3, 65.4, 51.1, 48.5, 47.6, 47.5, 47.4, 47.2, 47.0. ¹H NMR of 11 (500 MHz, MeOD) δ 7.83-7.62 (m, 3H), 7.32-7.08, (m, 2H) 4.71 (m, 1H, H-1), 4.98 (d, J=3.65 (dd, J₁=4.95 Hz, J₂=10.5 Hz, 1H, H-5), 3.54 (m, 1H, H-4), 3.46 (m, 1H, H-5), 3.35 (m, 1H, H-3), 2.20 (m, 1H, H-2), 1.75 (m, 1H, H-2); ¹³C NMR (500 MHz, MeOD) δ 197.0, 164.4, 161.0, 160.7, 136.5, 123.7, 121.1, 118.8, 117.2, 95.1, 66.8, 66.5, 62.0, 57.7, 49.6, 47.5, 47.4, 47.2, 47.0, 46.8, 46.7, 46.5, 32.6, 3.7 HRESI-MS [M+Na]⁺; calculated 408.1059, found 408.1018.

### Example 4

### Preparation of Hexose-Pyranoside AEGs 13-16, Effective Against DOX-Resistant Cancer Cell Lines

The synthesis of the hexose-pyranoside-Aloe-Emodin Glycoside (AEG) derivatives 13-16, that have been found to be effective against a variety of doxorubicin-resistant cancer cell lines is demonstrated in scheme 9.

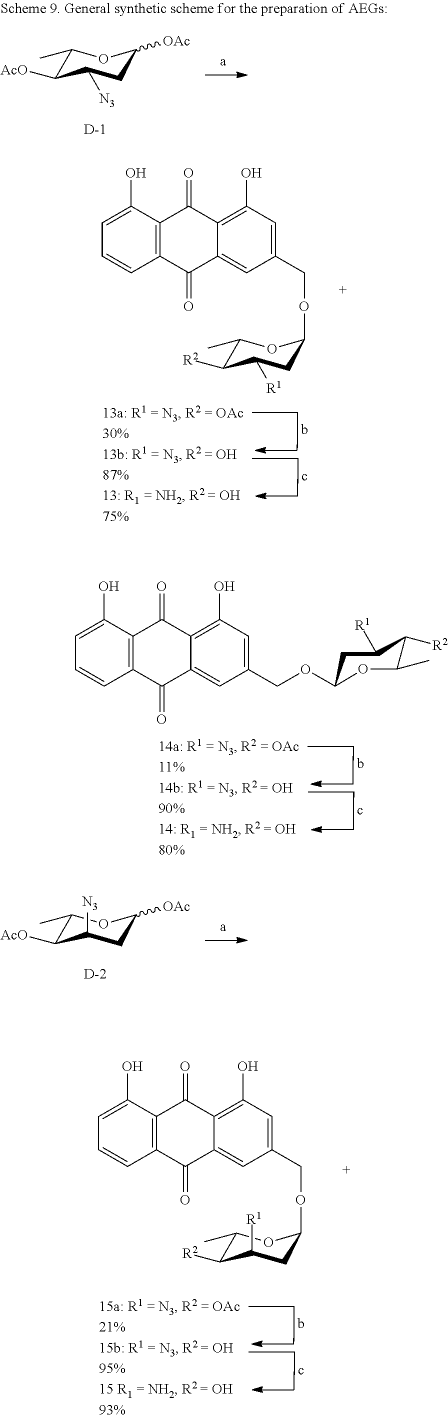

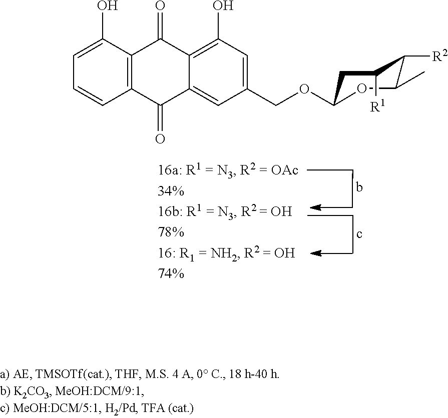

Generally, two 2,3,6-trideoxy-3-amino-L-glycosyl acetate donors: Acosamine D-1 and ristosamine D-2 which vary in the absolute configuration of their C-3 azide (Scheme 9), were prepared in three synthetic steps from commercially available 3,4-di-O-acetyl-L-rhamnal.^{[33,34]} Lewis acid catalyzed activation of the glycosyl acetates and AE in THF gave anomeric mixtures of AEGs 13a-16a which were readily separated by reverse phase HPLC.^{[19]} Removal of the acetyl groups under mild basic conditions gave AEGs 13a-16a which were purified by size exclusion column chromatography on Sephadex LH-20. The azido groups were transformed to the corresponding free amines by a final catalytic hydrogenation step and reverse phase HPLC purification gave the final AEGs 13-16 in good yields. AEGs13-16 represent all four combinations of two structural descriptors: the glycosidic linkage (α or β) and the carbohydrate C-3 amine absolute configuration (axial or equatorial), therefore enabling a structure activity relationship (SAR) study for these two features. All compounds were fully characterized by ¹H, ¹³C NMR and HRMS.

### Detailed Synthetic Procedures:

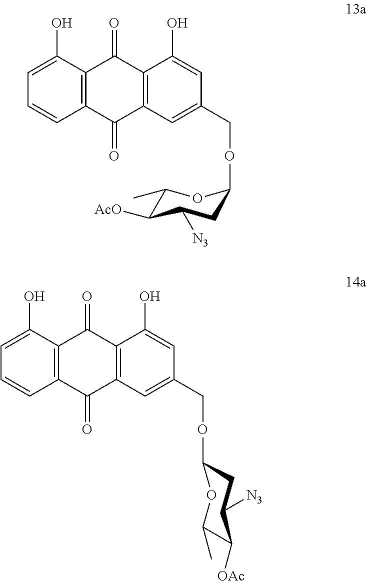

AEGs 13a and 14a: Acosamine glycosyl donor D-1 (295.0 mg, 1.15 mmol) and AE (283.4 mg, 1.05 mmol) in dry THF (6.0 ml) were added flame dried molecular sieves (4 Å, 400 mg) and stirred under argon atmosphere at ambient temperature for 20 min. The reaction mixture was then cooled to 0° C., added trimethylsilyl trifluoromethanesulfonate (60 μL, 0.33 mmol). Reaction progress was monitored by TLC (70% petroleum ether, 30% ethyl acetate) and indicated the formation of an anomeric mixture of products (α-anomer 13a R_{f}=0.71, (β-anomer 14a R_{f}=0.57). Upon completion (40 h at 0° C.) the reaction was quenched by trimethylamine (60 μL) and the crude was filtered through a small plug of celite. The products were isolated by reverse-phase HPLC using a Phenomenex Luna axia 5 μm C-18 (250 mm×21.20 mm) column at a flow rate of 20.0 mL/min. The HPLC solvents were A: H₂O (0.1% TFA) and B: ACN (0.1% TFA). The elution gradient was 80% B for 2 min followed by 80-100% B over 20 min and product elution was monitored at 256 nm by a UV detector. The product retention times were 9.4 minutes for the (α-anomer 13a and 8.8 minutes for the β-anomer 14a. Fractions containing the pure product were concentrated under reduced pressure to yield α-anomer 13a (162.4 mg, 0.35 mmol), β-anomer 14a (66.3 mg, 0.14 mmol) and a mixture of both anomers (229.9 mg, 0.49 mmol). The total isolated yield of the reaction was 43% with an α:β ratio of 1:3 as indicated by HPLC.

¹H NMR (500 MHz, CDCl₃) for AEG 13a δ: 12.01 (s, 1H, OH), 11.99 (s, 1H, OH) 7.77 (d, J=7.5 Hz, 1H, H-5′), 7.67 (s, 1H, H-4), 7.62 (t, J=7.8 Hz, 1H, H-6), 7.22 (m, 2H, H-2, H-7′), 4.93 (bd, J=3.2 Hz, 1H, H-1), 4.68 (d, J=13.6 Hz, 1H, H-15′), 4.64 (dd, J₁=J₂=9.8 Hz, 1H, H-4), 4.48 (d, J=13.7 Hz, 1H, H-15′), 3.87 (ddd, J₁=4.9, J₂=9.9 J₃=12.3 Hz, 1H, H-3), 3.75 (dq, J₁=6.3, J₂=9.6 Hz, 1H, H-5), 2.22 (bdd, J₁=4.9, J₂=13.3 Hz, 1H, H-2 eq), 2.06 (s, 3H, COCH₃), 1.74 (dd, J₁=3.5, J₂=12.9 Hz, 1H, H-2ax), 1.11 (d, J=6.3 Hz, 3H, H-6). ¹³C NMR (125.7 MHz, CDCl₃) for AEG 13a δ: 192.1 (C-9′), 181.1 (C-10′), 169.6 (COCH₃), 162.3, 162.1, 147.6 (C-3′), 136.8 (C-6′), 133.2, 133.0, 124.3, 121.7, 119.6, 117.9, 115.2, 114.5, 95.7 (C-1), 74.8, 67.3, 65.9, 57.0, 34.5 (C-2), 20.6 (COCH₃), 16.9 (C-6). Positive HRESIMS, m/z calcd 490.1226 for C₂₃H₂₁N₃O₈Na, found 490.1231 [M+Na]⁺.

¹HNMR (500 MHz, CDCl₃) for AEG 14a δ: 11.94 (s, 1H, OH), 11.93 (s, 1H, OH) 7.72 (d, J=7.4 Hz, 1H, H-5′), 7.64 (s, 1H, H-4′), 7.59 (t, J=7.9 Hz, 1H, H-6′), 7.20 (m, 2H, H-2′, H-7′), 4.88 (d, J=13.7 Hz, 1H, H-15′), 4.63 (dd, J₁=J₂=9.6 Hz, 1H, H-4), 4.58 (d, J=13.7 Hz, 1H, H-15′), 4.56 (dd, J₁=1.8, J₂=9.7 Hz, 1H, H-1), 3.48 (ddd, J₁=5.0, J₂=9.8, J₃=12.8 Hz, 1H, H-3), 3.40 (dq, J₁=6.2, J₂=9.5 Hz, 1H, H-5), 2.27 (ddd, J₁=1.4, J₂=4.8, J₃=12.9 Hz, 1H, H-2 eq), 2.06 (s, 3H, OCH₃), 1.72 (ddd, J₁=J₂=9.7, J₃=12.7 Hz, 1H, H-2ax), 1.18 (d, J₁=6.2 Hz, 3H, H-6). ¹³C NMR (100.6 MHz, CDCl₃) for AEG 14a δ: 193.3 (C-9′), 182.3 (C-10′), 170.7 (COCH₃), 163.5, 163.2, 148.9 (C-3′), 137.9 (C-6′), 134.3, 134.2, 125.4, 122.9, 120.8, 119.1, 116.5, 115.7, 99.5 (C-1), 75.6, 71.7, 69.9, 60.4, 36.8 (C-2), 21.6 (COCH₃), 18.2 (C-6). Positive HRESIMS, m/z calcd 490.1226 for C₂₃H₂₁N₃O₈Na, found 490.1224 [M+Na]⁺.

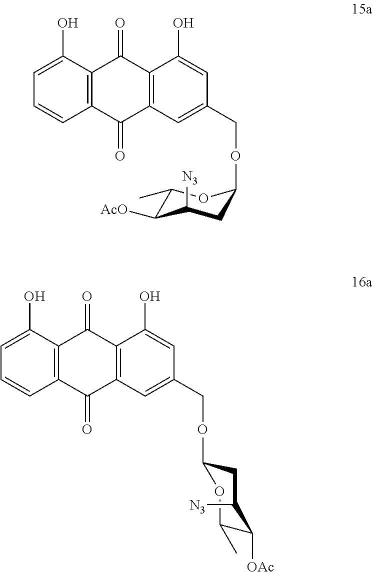

AEGs 15a and 16a: Ristosamine glycosyl donor D-2 (295.0 mg, 1.15 mmol) and AE (283.4 mg, 1.05 mmol) in dry THF (6.0 ml) were added flame dried molecular sieves (4 Å, 400 mg) and stirred under argon atmosphere at ambient temperature for 20 min. The reaction mixture was then cooled to 0° C., added trimethylsilyl trifluoromethanesulfonate (60 μL, 0.33 mmol). Reaction progress was monitored by TLC (70% petroleum ether, 30% ethyl acetate) and indicated the formation of an anomeric mixture of products (α-anomer 15a R_{f}=0.51, β-anomer 16a R_{f}=0.69). Upon completion (18 h at 0° C.) the reaction was quenched by trimethylamine (60 μL) and the crude was filtered through a small plug of celite. The products were isolated by reverse-phase HPLC using a Phenomenex Luna axia 5 μm C-18 (250 mm×21.20 mm) column at a flow rate of 20.0 mL/min. The HPLC solvents were A: H₂O (0.1% TFA) and B: ACN (0.1% TFA). The elution gradient was 80% B for 2 min followed by 80-100% B over 20 min and product elution was monitored at 256 nm by a UV detector. The product retention times were 8.1 minutes for the α-anomer 15a and 9.4 minutes for the β-anomer 16a. Fractions containing the pure product were concentrated under reduced pressure to yield the pure α-anomer 15a (113.4 mg, 0.24 mmol), the pure β-anomer 16a (182.7 mg, 0.39 mmol) and a mixture of both anomers (373.1 mg, 0.80 mmol). The total isolated yield of the reaction was 69% with an α:β ratio of 1:1 as indicated HPLC. ¹H NMR (500 MHz, CDCl₃) for AEG 15a δ: 12.02 (s, 1H, OH), 12.01 (s, 1H, OH) 7.78 (d, J=7.4 Hz, 1H, H-5′), 7.74 (s, 1H, H-4′), 7.62 (t, J=8.0 Hz, 1H, H-6′), 7.32 (s, 1H, H-2′), 7.24 (d, J=8.4 Hz, 1H, H-7′), 4.85 (d, J=4.0 Hz, 1H, H-1), 4.77 (d, J=14.0 Hz, 1H, H-15′), 4.62 (dd, J₁=3.5, J₂=9.6 Hz, 1H, H-4), 4.53 (d, J=14.0 Hz, 1H, H-15′), 4.17 (dq, J₁=6.3, J₂=9.5, 1H, H-5), 4.12 (ddd, J₁=J₂=J₃=3.5 Hz, 1H, H-3), 2.20 (bdd, J₁=3.0, J₂=14.9 Hz, 1H, H-2 eq), 2.07 (s, 3H, OCH₃), 2.05 (ddd, J₁=J₂=4.2, J₃=14.7 Hz, 1H, H-2ax), 1.11 (d, J=6.3 Hz, 3H, H-6). ¹³C NMR (100.6 MHz, CDCl₃) for AEG 15a δ: 193.4 (C-9′), 182.4 (C-10′), 170.9 (COCH₃), 163.6, 163.3, 149.5 (C-3′), 137.9 (C-6′), 134.4 (C-11′, C-14′), 125.4, 122.9, 120.8, 119.2, 116.6, 115.7, 95.9 (C-1), 74.5, 68.7, 63.0, 56.3, 30.4 (C-2), 21.5 (COCH₃), 18.0 (C-6). Positive HRESIMS, m/z calcd 490.1226 for C₂₃H₂₁N₃O₈Na, found 490.1226 [M+Na]⁺. ¹H NMR (500 MHz, CDCl₃) for AEG 16a δ: 12.01 (s, 1H, OH), 11.99 (s, 1H, OH) 7.77 (d, J=7.5 Hz, 1H, H-5′), 7.70 (s, 1H, H-4′), 7.63 (t, J=8.0 Hz, 1H, H-6′) 7.23 (d, J=7.9 Hz, 1H, H-7′), 7.19 (s, 1H, H-2′), 4.89 (d, J=13.6 Hz, 1H, H-15′), 4.77 (dd, J₁=2.0, J₂=8.9 Hz, 1H, H-1), 4.64 (dd, J₁=3.4, J₂=9.2 Hz, 1H, H-4), 4.58 (d, J=13.6 Hz, 1H, H-15′), 4.15 (ddd, J₁=J₂=J₃=3.5 Hz, 1H, H-3), 3.93 (dq, J₁=6.3, J₂=9.2 Hz, 1H, H-5), 2.08 (ddd, J₁=2.2, J₂=4.0, J₃=12.0 Hz, 1H, H-2 eq), 2.07 (s, 3H, OCH₃), 1.87 (ddd, J₁=3.4, J₂=9.0, J₃=12.4 Hz, 1H, H-2ax), 1.19 (d, J=6.4 Hz, 3H, H-6) ¹³C NMR (125.7 MHz, CDCl₃) for AEG 16a δ: 192.2 (C-9′), 181.2 (C-10′), 169.6 (COCH₃), 162.3, 162.0, 148.0 (C-3′), 136.7 (C-6′), 133.1 (C-11′, C-14′), 124.2, 121.8, 119.6, 118.0, 115.4, 114.5, 96.7 (C-1), 73.8, 68.7, 67.8, 57.1, 34.8 (C-2), 20.2 (COCH₃), 17.3 (C-6). Positive HRESIMS, m/z calcd 490.1226 for C₂₃H₂₁N₃O₈Na, found 490.1225 [M+Na]⁺.

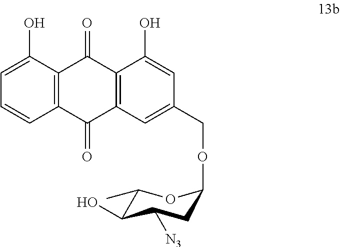

AEG 13b: AEG 13a (160.0 mg, 0.34 mmol) in MeOH:DCM/9:1 (5 mL) was added K₂CO₃ (50 mg, 0.36 mmol) and stirred at ambient temperature. Monitoring of the reaction by ESIMS indicated the disappearance of the starting material ([M−H]⁻, m/z 466.5) and formation of AEG 13b ([M−H]⁻, m/z 424.5). After 20 h acetic acid was added dropwise until the crimson red solution turned yellow. The volume of the crude mixture was reduced under vacuum to 1 mL and separated on by size-exclusion chromatography (Sephadex LH-20 loaded on a 700 mm length, 11.5 mm diameter column). The column was loaded and eluted with MeOH/DCM (1:1). Fractions containing the pure product were concentrated to yield AEG 13b as yellow powder (126.6 mg, 87%). ¹H NMR (500 MHz, CDCl₃) for AEG 13b δ: 12.01 (s, 1H, OH), 12.00 (s, 1H, OH) 7.77 (d, J=7.3 Hz, 1H, H-5′), 7.70 (s, 1H, H-4′), 7.62 (t, J=7.8 Hz, 1H, H-6′), 7.25-7.22 (m, 2H, H-2′, H-7′), 4.92 (bd, J=3.2 Hz, 1H, H-1), 4.70 (d, J=13.7 Hz, 1H, H-15′), 4.49 (d, J=13.7 Hz, 1H, H-15′), 3.77 (ddd, J₁=4.9, J₂=9.5, J₃=12.3 Hz, 1H, H-3), 3.66 (dq, J=6.2, J=9.2 Hz, 1H, H-5), 3.12 (dd, J₁=J₂=9.4 Hz, 1H, H-4), 2.22 (bdd, J₁=4.9, J₂=13.2 Hz, 1H, H-2 eq), 1.72 (ddd, J₁=3.6, J₂=J₃=12.9 Hz, 1H, H-2ax), 1.25 (d, J=6.3 Hz, 3H, H-6). ¹³C NMR (125.7 MHz, CDCl₃) for AEG 13b δ: 192.1 (C-9′), 181.1 (C-10′), 162.3, 162.1, 147.8 (C-3′), 136.7 (C-6′), 133.2, 133.1, 124.2, 121.7, 119.6, 118.0, 115.2, 114.5, 95.8 (C-1), 75.3, 67.7, 67.2; 59.7, 34.3 (C-2), 17.2 (C-6). Positive HRESIMS, m/z calcd 448.1121 for C₂₁H₁₉N₃O₇Na, found 448.1119 [M+Na]⁺.

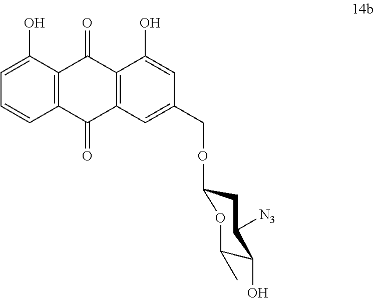

AEG 14b: AEG 14a (58.3 mg, 0.12 mmol) in MeOH:DCM/9:1 (5 mL) was added K₂CO₃ (50 mg, 0.36 mmol) and stirred at ambient temperature. Monitoring of the reaction by ESIMS indicated the disappearance of the starting material ([M−H]⁻, m/z 466.5) and formation of AEG 14b ([M−H]⁻, m/z 424.5). After 20 h acetic acid was added dropwise until the crimson red solution turned yellow. The volume of the crude mixture was reduced under vacuum to 1 mL and separated on by size-exclusion chromatography (Sephadex LH-20 loaded on a 700 mm length, 11.5 mm diameter column). The column was loaded and eluted with MeOH/DCM (1:1). Fractions containing the pure product were concentrated to yield AEG 14b as yellow powder (47.8 mg, 90%). ¹H NMR (500 MHz, CDCl₃) for AEG 14b δ: 12.01 (s, 1H, OH), 11.97 (s, 1H, OH) 7.77 (d, J=7.4 Hz, 1H, H-5′), 7.71 (s, 1H, H-4′), 7.62 (t, J=7.9 Hz, 1H, H-6′), 7.20 (m, 2H, H-2′, H-7′), 4.91 (d, J=13.7 Hz, 1H, H-15′), 4.61 (d, J=13.7 Hz, 1H, H-15′) 4.57 (dd, J₁=1.6, J₂=9.6 Hz, 1H, H-1), 3.37 (ddd, J₁=4.9, J₂=9.4, J₃=12.4 Hz, 1H, H-3), 3.30 (dq, J₁=6.1, J₂=9.0 Hz, 1H, H-5), 3.12 (ddd, J₁=3.5, J₂=J₃=9.1 Hz, 1H, H-4), 2.26 (ddd, J₁=1.7, J₂=4.9, J₃=12.8 Hz, 1H, H-2 eq), 2.16 (d, J=3.7 Hz, 1H, OH in C-4), 1.70 (ddd, J₁=9.7, J₂=J₃=12.6, Hz, 1H, H-2ax) 1.31 (d, J=6.1 Hz, 3H, H-6). ¹³C NMR (100.6 MHz, CDCl₃) for AEG 14b δ: 207.7 (C-9′), 182.4 (C-10′), 163.6, 163.3, 149.1 (C-3′), 137.9 (C-6′), 134.4, 134.3, 125.5, 123.0, 120.8, 119.3, 116.6, 115.8, 99.5 (C-1), 76.2, 73.4, 69.9, 63.2, 31.6 (C-2), 18.5 (C-6). Positive HRESIMS, m/z calcd 448.1121 for C₂₁H₁₉N₃O₇Na, found 448.1123 [M+Na]⁺.

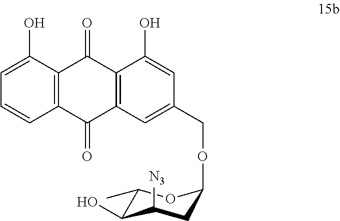

AEG 15b: AEG 15a (113.4 mg, 0.24 mmol) in MeOH:DCM/9:1 (5 mL) was added K₂CO₃ (50 mg, 0.36 mmol) and stirred at ambient temperature. Monitoring of the reaction by ESIMS indicated the disappearance of the starting material ([M−H]⁻, m/z 466.5) and formation of AEG 15b ([M−H]⁻, m/z 424.5). After 20 h acetic acid was added dropwise until the crimson red solution turned yellow. The volume of the crude mixture was reduced under vacuum to 1 mL and separated on by size-exclusion chromatography (Sephadex LH-20 loaded on a 700 mm length, 11.5 mm diameter column). The column was loaded and eluted with MeOH/DCM (1:1). Fractions containing the pure product were concentrated to yield AEG 15b as yellow powder (98.0 mg, 95%). ¹H NMR (500 MHz, CDCl₃) for AEG 15b δ: 12.02 (s, 1H, OH), 12.01 (s, 1H, OH) 7.77 (d, J=7.4 Hz, 1H, H-5′), 7.75 (s, 1H, H-4′), 7.62 (t, J=8.0 Hz, 1H, H-6′), 7.30 (s, 1H, H-2′), 7.25 (d, J=8.6 Hz, 1H, H-7′), 4.86 (d, J=4.0 Hz, 1H, H-1), 4.77 (d, J=14.0 Hz, 1H, H-15′), 4.51 (d, J=14.0 Hz, 1H, H-15′), 4.07 (ddd, J₁=J₂=J₃=3.6 Hz, 1H, H-3), 3.84 (dq, J₁=6.3, J₂=9.3 Hz, 1H, H-5), 3.29 (dd, J₁=3.5, J₂=9.2 Hz, 1H, H-4), 2.31 (bdd, J₁=2.3, J₂=15.1 Hz, 1H, H-2 eq), 2.05 (ddd, Jᵣ=J₂=4.2, J₃=15.2 Hz, 1H, H-2ax), 1.20 (d, J=6.3 Hz, 3H, H-6) ¹³C NMR (100.6 MHz, CDCl₃) for AEG 15b δ: 193.4 (C-9′), 182.4 (C-10′), 163.6, 163.2, 149.7 (C-3′), 137.9 (C-6′), 134.3 (C-11′, C-14′), 125.4, 122.7, 120.8, 119.0, 116.6, 115.6, 95.7 (C-1), 72.6, 68.6, 65.6, 58.7, 32.8 (C-2), 18.2 (C-6). Positive HRESIMS, m/z calcd 448.1121 for C₂₁H₁₉N₃O₇Na, found 448.1118 [M+Na]⁺.

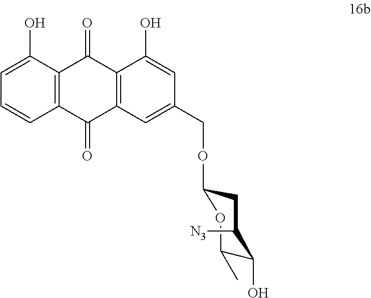

AEG 16b: AEG 16a (182.7 mg, 0.39 mmol) in MeOH:DCM/9:1 (5 mL) was added K₂CO₃ (50 mg, 0.36 mmol) and stirred at ambient temperature. Monitoring of the reaction by ESIMS indicated the disappearance of the starting material ([M−H]⁻, m/z 466.5) and formation of AEG 16b ([M−H]⁻, m/z 424.5). After 20 h acetic acid was added dropwise until the crimson red solution turned yellow. The volume of the crude mixture was reduced under vacuum to 1 mL and separated on by size-exclusion chromatography (Sephadex LH-20 loaded on a 700 mm length, 11.5 mm diameter column). The column was loaded and eluted with MeOH/DCM (1:1). Fractions containing the pure product were concentrated to yield AEG 16b as yellow powder (130.0 mg, 78%). ¹HNMR (500 MHz, CDCl₃) for AEG 16b δ: 12.00 (s, 1H, OH), 11.98 (s, 1H, OH) 7.75 (d, J=7.4 Hz, 1H, H-5′), 7.69 (s, 1H, H-4′), 7.61 (t, J=7.9 Hz, 1H, H-6′), 7.25-7.22 (m, 2H, H-2′, H-7′), 4.90 (d, J=13.7 Hz, 1H, H-15′), 4.78 (dd, J₁=2.0, J₂=9.0 Hz, 1H, H-1), 4.57 (d, J=13.7 Hz, 1H, H-15′), 4.06 (ddd, J₁=J₂=J₃=3.5 Hz, 1H, H-3), 3.63 (dq, J₁=6.3, J₂=8.9 Hz, 1H, H-5), 3.39 (dd, J₁=3.0, J₂=8.6 Hz, 1H, H-4), 2.19 (ddd, J₁=2.2, J₂=3.7, J₃=14.0 Hz, 1H, H-2 eq), 1.87 (ddd, J₁=3.4, J₂=9.1, J₃=13.9 Hz, 1H, H-2ax), 1.27 (d, J=6.3 Hz, 3H, H-6). ¹³C NMR (100.6 MHz, CDCl₃) for AEG 16b δ: 192.2 (C-9′), 181.2 (C-10′), 162.3, 162.0, 148.0 (C-3′), 136.7 (C-6′), 133.1 (C-11′, C-14′), 124.2, 121.8, 119.6, 118.0, 115.3, 114.4, 96.6 (C-1), 72.0, 70.2, 68.7, 60.3 (C-5 or C-15′ or C-4 or C-3), 34.7 (C-2), 17.4 (C-6). Positive HRESIMS, m/z calcd 448.1121 for C₂₁H₁₉N₃O₇Na, found 448.1122 [M+Na]⁺.

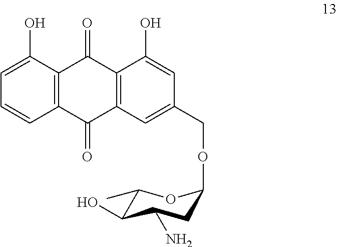

AEG 13: AEG 13b (20.6 mg, 48 μmol) dissolved in MeOH:DCM/5:1 (×mL) added palladium on carbon (10 mg), trifluoroacetic acid (10 μL) and stirred at ambient temperature under a hydrogen balloon. Monitoring of the reaction by ESIMS indicated the disappearance of the starting material ([M−H]⁻, m/z 424.5) and formation of AEG 13 ([M−H]⁻, m/z 398.5). After 15 min, the mixture was filtered (PHENEX PTFE Membrane, 0.2 μm, 15 mm Syringe Filters) and purified by HPLC using Phenomenex Luna C18 HPLC column at a flow rate of 20 mL/min. The HPLC solvents were A: H₂O (0.1% TFA) and B: ACN (0.1% TFA). The elution gradient was 50% B for 2 min followed by 50-100% B over 15 min. Product elution was monitored at 256 nm. The product was detected after 3.9 min. Fractions containing the pure product were concentrated under vacuum, dissolved in H₂O, and freeze-dried to yield AEG 13 as a yellow powder (10.7 mg, 75%). ¹H NMR (500 MHz, MeOH-d₄) for AEG 13 δ: 7.66-7.75 (m, 3H, H-4′, H-5′, H-6′), 7.27 (d, J=8.1 Hz, 1H, H-7′), 7.24 (s, 1H, H-2′), 5.00 (bd, J=3.0 Hz, 1H, H-1), 4.72 (d, J=13.7 Hz, 1H, 1′-15′), 4.57 (d, J=13.7 Hz, 1H, H-15″), 3.64 (dq, J₁=6.2, J₂=9.1 Hz, 1H, H-5), 3.40 (ddd, J₁=4.5, J₂=10.1, J₃=12.7 Hz, 1H, H-3), 3.09 (dd, J₁=J₂=9.6 Hz, 1H, H-4), 2.24 (bdd, J₁=4.5, J₂=12.9 Hz, 1H, H-2 eq), 1.82 (ddd, J₁=3.5, J₂=J₃=12.8 Hz, 1H, H-2ax), 1.21 (d, J=6.2 Hz, 3H, H-6). ¹³C NMR (125.7 MHz, MeOH-d₄) for AEG 13 δ: 192.0 (C-9′), 181.0 (C-10′), 161.9, 161.7, 148.1 (C-3′), 136.5 (C-6′), 133.2, 133.1, 123.7, 121.2, 118.8, 117.3, 115.2, 114.4, 95.1 (C-1), 72.4, 68.2, 67.0, 49.5, 34.3 (C-2), 17.2 (C-6). Positive HRESIMS, m/z calcd 400.1396 for C₂₁H₂₂NO₇, found 400.1396 [M+H]⁺.

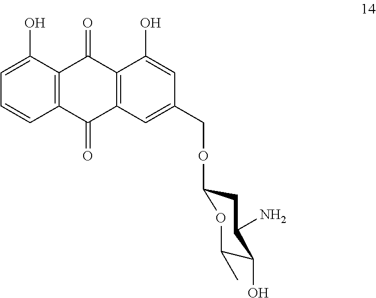

AEG 14: AEG 14b (17.0 mg, 40 μmol) dissolved in MeOH:DCM/5:1 (×mL) added palladium on carbon (10 mg), trifluoroacetic acid (10 μL) and stirred at ambient temperature under a hydrogen balloon. Monitoring of the reaction by ESIMS indicated the disappearance of the starting material ([M−H]⁻, m/z 424.5) and formation of AEG 14 ([M−H]⁻, m/z 398.5). After 15 min, the mixture was filtered (PHENEX PTFE Membrane, 0.2 μm, 15 mm Syringe Filters) and purified by HPLC using Phenomenex Luna C18 HPLC column at a flow rate of 20 mL/min. The HPLC solvents were A: H₂O (0.1% TFA) and B: ACN (0.1% TFA). The elution gradient was 50% B for 2 min followed by 50-100% B over 15 min. Product elution was monitored at 256 nm. The product was detected after 4.0 min. Fractions containing the pure product were concentrated under vacuum, dissolved in H₂O, and freeze-dried to yield AEG 14 as a yellow powder (6.1 mg, 80%). ¹H NMR (500 MHz, MeOH-d₄) for AEG 14 δ: 7.65-7.74 (m, 3H, H-4′, H-5′, H-6′), 7.26 (d, J=8.0 Hz, 1H, H-7′), 7.21 (s, 1H, H-2′), 4.88 (d, J=13.7 Hz, 1H, H-15), 4.72 (dd, J₁=1.9, J₂=9.4 Hz, 1H, H-1), 4.66 (d, J=13.7 Hz, 1H, H-15′), 3.33 (dq, J₁=6.2, J₂=8.8 Hz, 1H, H-5), 3.14 (ddd, J₁=4.6, J₂=9.9, J₃=12.3 Hz, 1H, H-3), 3.07 (dd, J₁=J₂=9.3 Hz, 1H, H-4), 2.27 (ddd, J₁=1.7, J₂=4.4, J₃=12.1 Hz, 1H, H-2 eq), 1.67 (ddd, J₁=9.5, J₂=J₃=12.3 Hz, 1H, H-2ax), 1.27 (d, J=6.2 Hz, 3H, H-6) ¹³C NMR (125.7 MHz, MeOH-d₄) for AEG 14 δ: 192.0 (C-9′), 180.9 (C-10′), 161.8, 161.7, 148.2 (C-3′), 136.5 (C-6′), 133.1, 133.0, 123.7, 121.1, 118.8, 117.3, 115.2, 114.3, 98.1 (C-1), 72.6, 72.2, 68.5, 51.6, 34.5 (C-2), 16.0 (C-6). Positive HRESIMS, m/z calcd 400.1396 for C₂₁H₂₂NO₇, found 400.1393 [M+H]⁺.

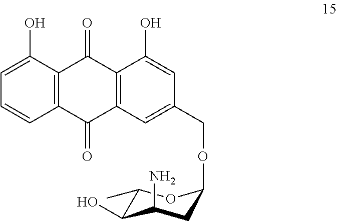

AEG 15: AEG 15b (20.0 mg, 47 μmol) dissolved in MeOH:DCM/5:1 (×mL) added palladium on carbon (10 mg), trifluoroacetic acid (10 μL) and stirred at ambient temperature under a hydrogen balloon. Monitoring of the reaction by ESIMS indicated the disappearance of the starting material ([M−H]⁻, m/z 424.5) and formation of AEG 15 ([M−H]⁻, m/z 398.5). After 15 min, the mixture was filtered (PHENEX PTFE Membrane, 0.2 μm, 15 mm Syringe Filters) and purified by HPLC using Phenomenex Luna C18 HPLC column at a flow rate of 20 mL/min. The HPLC solvents were A: H₂O (0.1% TFA) and B: ACN (0.1% TFA). The elution gradient was 50% B for 2 min followed by 50-100% B over 15 min. Product elution was monitored at 256 nm. The product was detected after 4.0 min. Fractions containing the pure product were concentrated under vacuum, dissolved in H₂O, and freeze-dried to yield AEG 15 as a yellow powder (17.4 mg, 93%). ¹H NMR (500 MHz, MeOH-d₄) for AEG 15 δ: 7.65-7.73 (m, 3H, 1′-4′, H-5′, H-6′), 7.23-7.29 (m, 2H, H-2′, H-7′), 4.91 (d, J=2.4 Hz, 1H, H-1), 4.76 (d, J=14.1 Hz, 1H, H-15′), 4.60 (d, J=14.1 Hz, 1H, H-15′), 3.82 (dq, J₁=6.1, J₂=9.6 Hz, 1H, H-5), 3.54 (ddd, J₁=J₂=J₃=3.6 Hz, 1H, H-3), 3.46 (dd, J₁=4.4, J₂=9.7 Hz, 1H, H-4), 2.16 (bd, J=15.3 Hz, 1H, H-2 eq), 2.10 (ddd, J₁=J₂=3.8, J₃=15.2 Hz, 1H, H-2ax), 1.24 (d, J=6.1 Hz, 3H, H-6)¹³C NMR (100.6 MHz, MeOH-d₄) for AEG 15 δ: 193.3 (C-9′), 182.3 (C-10′), 163.1, 163.0, 149.0 (C-3′), 137.8 (C-6′), 134.4, 134.3, 125.0, 122.8, 120.1, 118.7, 116.4, 115.7, 96.3 (C-1), 68.9, 68.5, 64.7, 49.8, 31.9 (C-2), 17.5 (C-6). Positive HRESIMS, m/z calcd 400.1396 for C₂₁H₂₂NO₇, found 400.1394 [M+H]⁺.

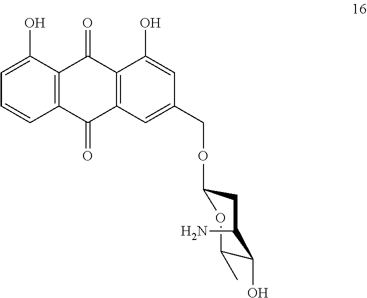

AEG 16: AEG 16b (18.0 mg, 42 μmol) dissolved in MeOH:DCM/5:1 (×mL) added palladium on carbon (10 mg), trifluoroacetic acid (10 μL) and stirred at ambient temperature under a hydrogen balloon. Monitoring of the reaction by ESIMS indicated the disappearance of the starting material ([M−H]⁻, m/z 424.5) and formation of AEG 16 ([M−H]⁻, m/z 398.5). After 15 min, the mixture was filtered (PHENEX PTFE Membrane, 0.2 μm, 15 mm Syringe Filters) and purified by HPLC using Phenomenex Luna C18 HPLC column at a flow rate of 20 mL/min. The HPLC solvents were A: H₂O (0.1% TFA) and B: ACN (0.1% TFA). The elution gradient was 50% B for 2 min followed by 50-100% B over 15 min. Product elution was monitored at 256 nm. The product was detected after 4.0 min. Fractions containing the pure product were concentrated under vacuum, dissolved in H₂O, and freeze-dried to yield AEG 16 as a yellow powder (11.2 mg, 74%). ¹H NMR (500 MHz, MeOH-d₄) for AEG 16 δ: 7.61-7.68 (m, 3H, H-4′, H-5′, H-6′), 7.23 (dd, J₁=2.9, J₂=6.6 Hz, 1H, H-7′), 7.17 (s, 1H, H-2′), 4.93 (dd, J₁=2.9, J₂=6.0 Hz, 1H, H-1), 4.84 (d, J=13.9 Hz, 1H, H-15′), 4.59 (d, J=13.9, 1H, H-15″Hz), 3.79 (dq, J₁=J₂=6.5, Hz, 1H, H-5), 3.72 (ddd, J₁=J₂=4.3, J₃=7.4 Hz, 1H, H-3), 3.59 (dd, J₁=4.1, J₂=6.0 Hz, 1H, H-4), 2.19 (ddd, J₁=2.9, J₂=7.1, J₃=13.9 Hz, 1H, H-2 eq), 1.99 (ddd, J₁=J₂=4.9, J₃=13.9 Hz, 1H, H-2ax), 1.31 (d, J=6.6 Hz, 3H, H-6)¹³C NMR (100.6 MHz, MeOH-d₄) for AEG 16 δ: 193.2 (C-9′), 182.0 (C-10′), 163.0, 162.9, 149.5 (C-3′), 137.7 (C-6′), 134.3, 134.2, 124.9, 122.3, 120.0, 118.4, 116.4, 115.5, 97.2 (C-1), 72.8, 69.0, 68.4, 49.1, 31.7 (C-2), 18.6 (C-6). Positive HRESIMS, m/z calcd 400.1396 for C₂₁H₂₂NO₇, found 400.1399 [M+H]⁺.

### Example 5

### Cytotoxicity Assay—Compounds 11 and 12

The cytotoxicity of doxorubicin (Dox), Aloe-Emodin (Alo), Aloe-Emodin connected through C-7 to alpha 2-deoxy-3-deoxy-3-amino-L-xylopyranose (E-1 or “compound 11”), and Aloe-Emodin connected through C-7 to beta 2-deoxy-3-deoxy-3-amino-L-xylopyranose (E-2 or “compound 12”), towards four cell lines representing four different histological types of cancer: Molt-4 (human T cell leukemia), B16 (murine melanoma), HCT 116 (human colon adenocarcinoma), and MCA 105 (murine fibrosarcoma), were compared.

The respective cell type at 1×10⁴ cells/well was incubated for 24 hours in 96-well plates and cell viability was determined using the 2,3-bis(2-methoxy-4-nitro-5-sulphophenyl)-2H-tetrazolium-5-carboxanilide (XTT) kit (Biological Industries, Israel). The assay is based on the ability of metabolically active cells to reduce the tetrazolium salt XTT to orange colored compounds of formazan. The dye formed is water soluble and the dye intensity was read at 490 nM with a VERSAmax microplate ELISA reader (Molecular Devices). Optical density is directly proportional to the number of living cells in culture. Cytotoxicity (%) was calculated in the following way: [(absorbance of control cells−absorbance of drug-treated cells)/absorbance of control cells]×100. Results shown are averages of 3 repeats ±SE.

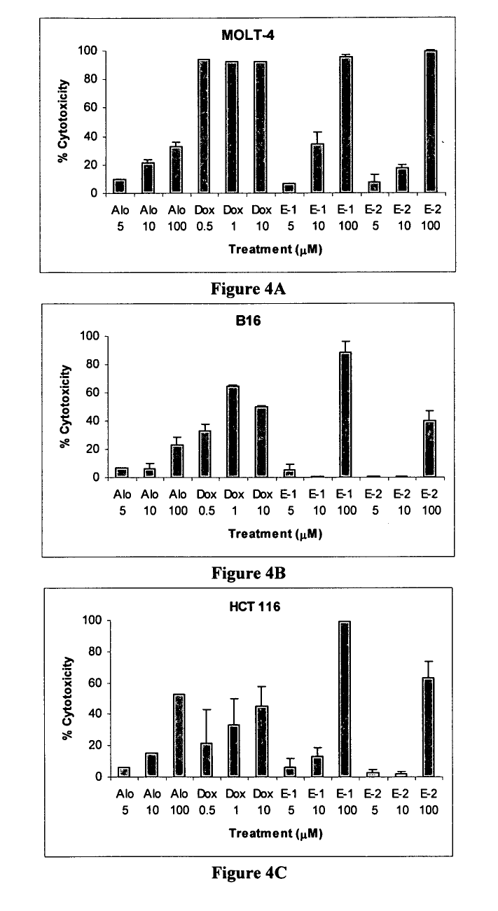

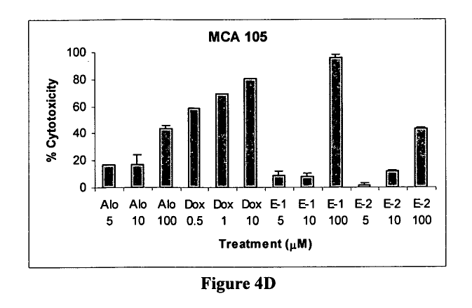

As can be seen in FIG. 4, the general outcome following 24 hours of incubation is similar for all four types of cancer cells: Dox is much more effective than the other compounds; E-1 at the highest concentration is much more effective than Alo, while E-2 exhibits a much weaker effect. Interestingly, it was also determined that while Dox, Alo and E-2, at up to 8 hours of incubation with Molt-4 cells induce only marginal toxicities (about 20%), E-1 (100 μM) at 4 hours of incubation already induces about 90% cytotoxicity. Over all, these preliminary results suggest that the beta glycosidic bond (such as the one in compound E-2) is less biologically active when compared to the alpha glycosidic bond (such as the one in compound E-1). This observation is in agreement with the fact that the glycosidic bond in all of the common anthracyclines has the alpha configuration thus facilitating better binding to the minor groove of the DNA. In addition, with these two analogs it was established that Aloe-Emodin functionalized by suitable carbohydrates at C-7, acquires cytotoxic features which are equivalent to those of commonly used anthracyclines. This fact makes Aloe-Emodin sugar analogs a promising model compound for the development of anthracycline derivatives with reduced toxic side effects.

### Example 6

### Antitumor Activity of AEGs 13-16 Against Doxorubicin Resistant Cell Lines

### A. Cytotoxicity Assay:

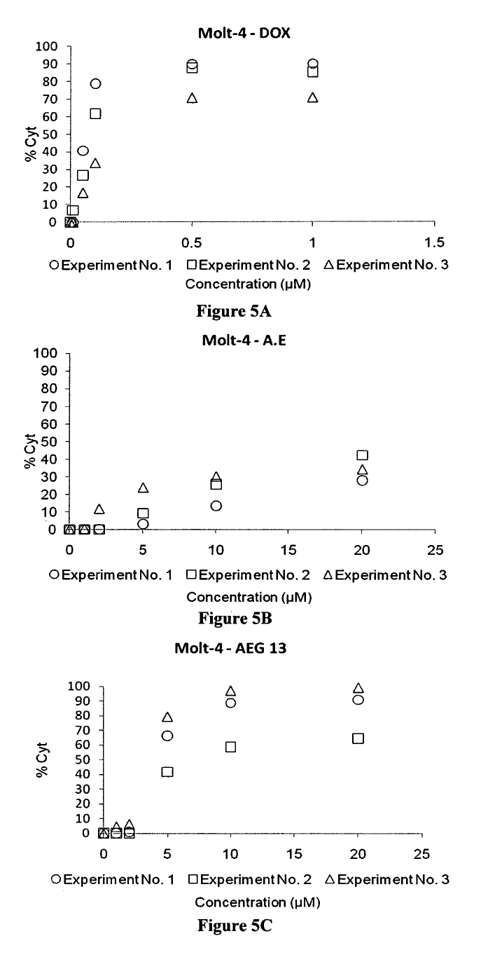

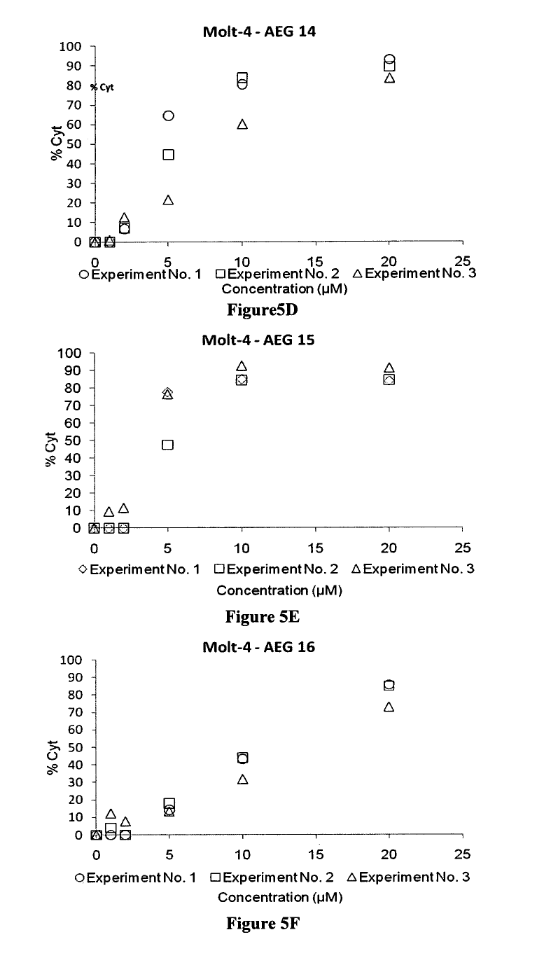

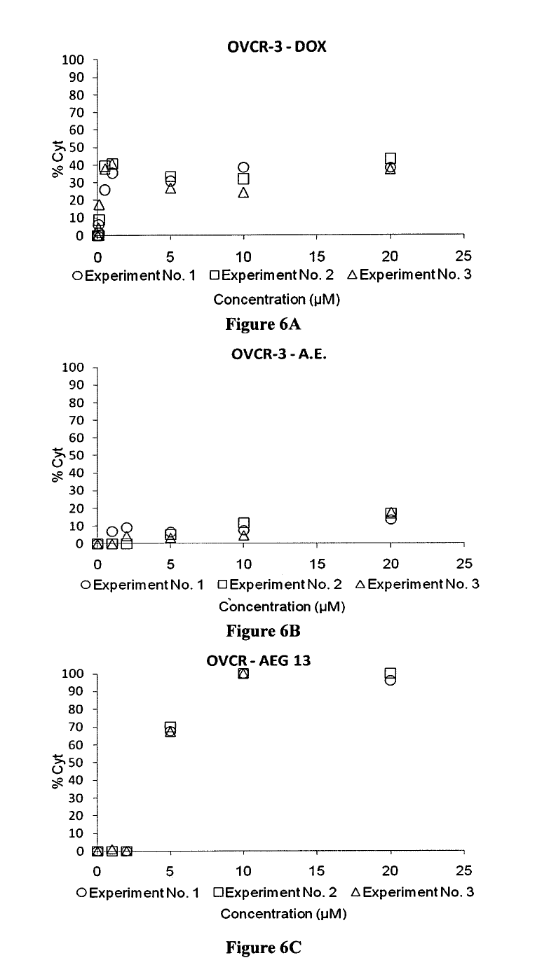

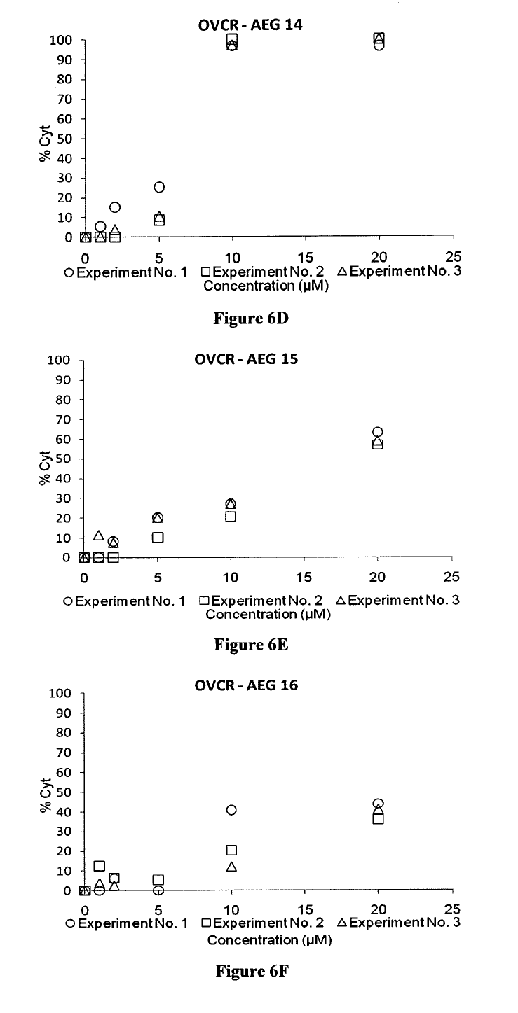

AEGs cytotoxicity was tested by determining the IC₅₀ values after a 24 hour incubation of cell lines representing leukemia, ovarian and breast cancers with several concentrations of AEGs 13-16 (Table 1 and FIGS. 5-8)^{[35]}. Leukemia representing MOLT-4 line exhibited high sensitivity towards DOX (IC₅₀=0.20±0.07 μM) (FIG. 5A). Compared to AE which had no effect on MOLT-4 cells even at 20 μM (100% viability) (FIG. 5B), AEGs 13-16 demonstrated improved activity with IC₅₀ values ranging between 5.8±1.3 μM for AEG 13 and 12.8±0.7 μM for AEG 16 (FIG. 5C-5F) Ovarian cancer line OVCAR-3 was less sensitive to DOX with 61% cell viability at 20 μM (FIG. 6A). For OVCAR-3, acosamine AEGs 13 and 14 demonstrated the most potent cytotoxic activity (IC₅₀=5.2±0.1 and 6.4±0.2 μM respectively) (FIG. 6C-6D) while AEG 15 was less active (IC₅₀=15.7±0.8 μM) (FIG. 6E) and 60% cell viability was detected for cells that were exposed to 20 μM of AEG 16 (FIG. 6F).

TABLE 1
IC-50 values after 24 h incubation with AEGs 1-4

| | MOLT-4 | OVCAR-3 | MCF-7 | NAR |
|---|---|---|---|---|
| DOX | 0.20 ± 0.07 | >20 | >20 | >100 |
| AE | >20 | >20 | >20 | >100 |
| AEG 13 | 5.8 ± 1.3 | 5.2 ± 0.1 | 7.1 ± 0.3 | 8.6 ± 0.6 |
| AEG 14 | 7.6 ± 1.6 | 6.4 ± 0.2 | 11.9 ± 0.6 | >100 |
| AEG 15 | 5.4 ± 0.4 | 15.7 ± 0.8 | 12.7 ± 1.0 | 18.0 ± 1.3 |
| AEG 16 | 12.8 ± 0.7 | >20 | >20 | 28.3 ± 2.3 |

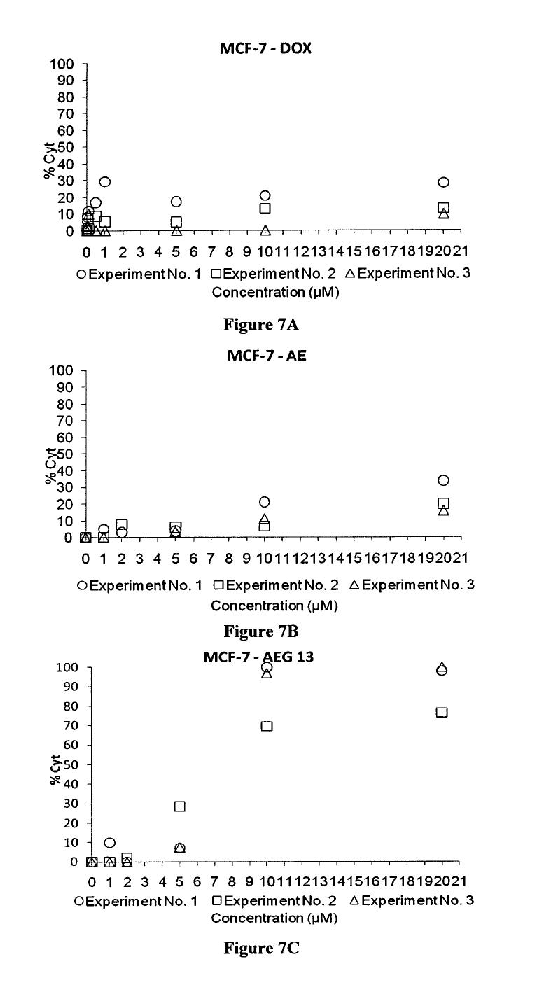

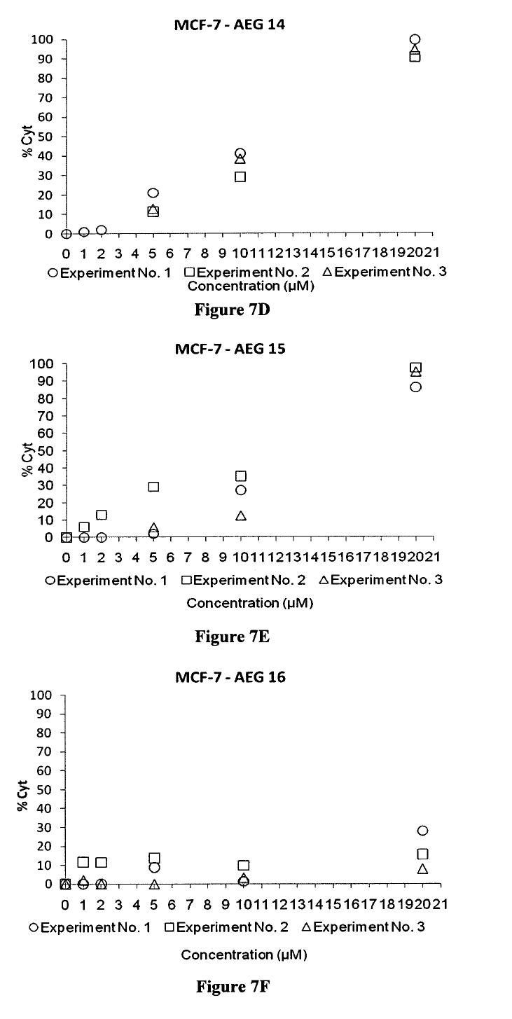

DOX resistant breast cancer line MCF-7 was poorly affected by the drug as well as by AE (83% and 77% viability at 20 μM respectively) (FIG. 7 A-F). As with OVCAR-3 and MOLT-4, AEG 13 was potent and the most active AEG (IC₅₀=7.1±0.3 μM) (FIG. 7C). AEGs 14 and 15 were mildly less active against OVCAR-3 cells (IC₅₀=11.9±0.6 and 12.7±1.0 respectively) (FIG. 7D-7E) while with AEG 16, 83% cell viability was observed at a concentration of 20 μM (FIG. 7F).

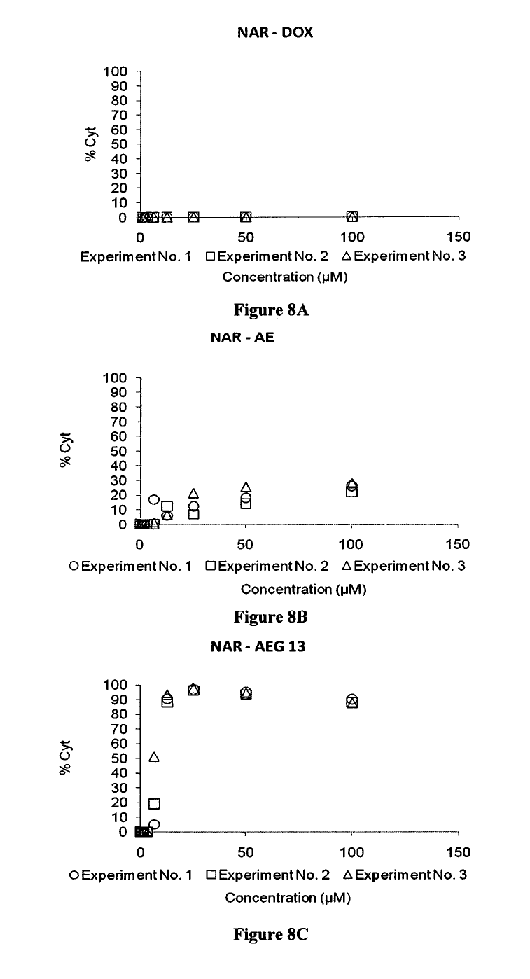

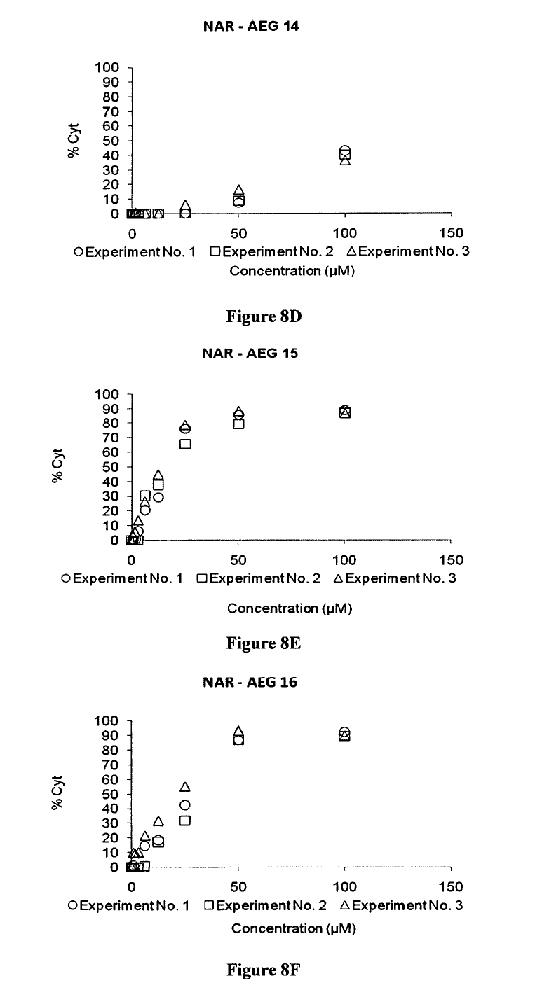

Finally, AEGs 13-16 were evaluated against DOX resistant ovarian cancer NAR cells in which resistance is conferred by overexpression of P-gp efflux pumps^{[36]} (FIG. 8 A-F) NAR cells were not affected at all by a high DOX concentration of a 100 μM (FIG. 8A), and AE (FIG. 8B) exhibited 78% viability at the same concentration. Once again, AEG 13 exhibited the most potent cytotoxicity (IC₅₀ of 8.6±0.6 μM) (FIG. 8C) which is at least two orders of magnitude improvement compared to DOX and AE. As compared with α-acosamine AEG 13, the β-acosamine AEG 14 was less active (65% viability at 100 μM) (FIG. 8D). For NAR cells, α-ristosamine AEG 15 was more active then the β-ristosamine AEG 16 (IC₅₀=18.0±1.3 and 28.3±2.3 μM respectively) (FIGS. 8E-8F).

Overall, the sugar attachment to AE resulted in improved cytotoxicity of the AEGs as compared with AE alone. For the tested cell lines, the combination of an α-glycosidic linkage and an equatorial carbohydrate C-3 amine in AEG 13 resulted with greater cytotoxic activity compared to that of the β-glycosidic linkage and carbohydrate axial C-3 amine in AEG 16, although both compounds displayed potent cytotoxic activity against doxorubicin resistant cell lines. The relative cytotoxic activity of AEGs 14 and 15 varied between the tested cell lines, with each displaying cytotoxic activity against the tested doxorubicin-resistant cell lines.

### B. Light Microscopy Study

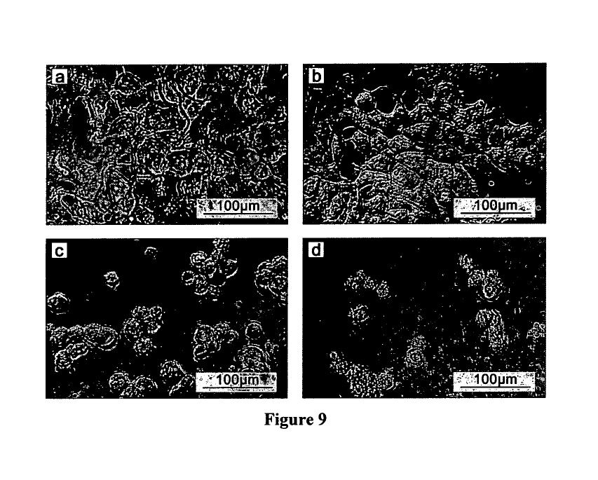

Light microscopy revealed that exposure to AEGs resulted in a decrease in cell numbers and a change in cells morphology (FIG. 9). For example, compared to untreated cells (FIG. 9a), cells that were pre-incubated with 20 μM AE were not affected in shape or numbers (FIG. 9b). Cells treated with DOX (20 μM, FIG. 9c) became more spherical in shape and smaller in size. The most significant effect was detected for cells which were pre-incubated with AEG 13 (20 μM), where a very small number of highly damaged cells was observed (FIG. 9d).

### C. DNA Intercalation Properties

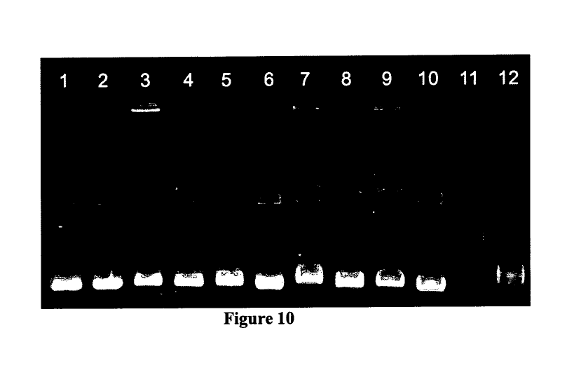

The DNA intercalation property of AEGs 13-16 was studied by applying the robust supercoiled plasmid DNA unwinding gel experiments protocol.^{[23,37,38]} Briefly, samples containing one of the AEGs 13-16, DOX or AE were pre-incubated with PBR322DNA plasmid, loaded on a 1% agarose gel, run for 4 hours at 70 volts and stained with ethidium bromide. At 200 μM, AE had no observable DNA shift effect (lane 2, FIG. 10) indicating its low DNA affinity. Compared to AE, an intense effect was observed for DOX at 200 μM (lane 11, FIG. 10). The effect of DOX was still significant at 20 μM (lane 12, FIG. 10). AEGs 13-16 caused a detectable DNA shift, at 200 μM yet no effect was observed at 20 μM. At 200 μM. AEG 13 (lane 3, FIG. 10) had the most significant effect, whereas a weak effect was detected for AEG 14 (lane 5, FIG. 10).

### D. Fluorescent Confocal Microscopy Study

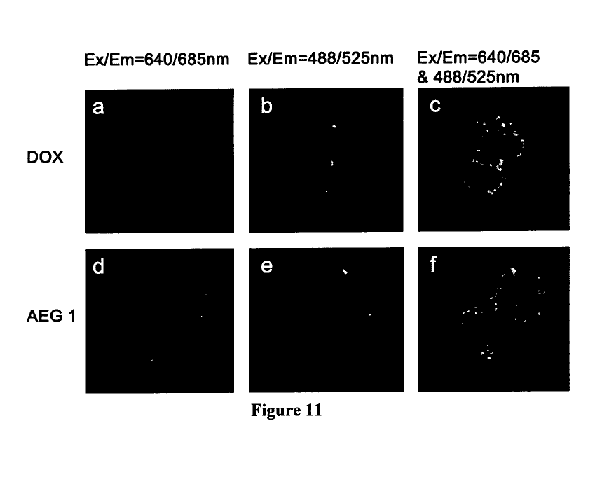

A possible explanation for the potency of the synthetic AEGs against. DOX resistant cells was provided using fluorescent confocal microscopy (FIG. 11). The cell permeability of the AEGs was tested on DOX resistant NAR cells, which were pre-incubated with either DOX or AEG 13 which was the most potent against this cell line. A two hour incubation time and a concentration of 5 μM of DOX and AEG 13 were chosen to avoid significant cell damage during the experiment. Cells were then fixated by paraformaldehyde and the plasma membrane was stained by carbocyanine tracer DiD (DiIC₁₈ (5)-DS). Fluorescent confocal microscopy (Ex/Em=488/525 nm for DOX and AEG 13 and Ex/Em=640/685 nm for DiIC₁₈ (5)-DS) indicated that DOX accumulated mainly in the plasma membrane (FIG. 11a-c). In cells pre-treated with AEG 13, an intracellular accumulation of the compound was observed (FIG. 11 d-f).

In conclusion, a novel class of AEGs targeting anthracycline resistant tumor cells was designed and synthesized. All of the AEGs exhibited improved cytotoxic activity on several tumor cell lines representing cancers with different levels of anthracycline resistance. A comparison of AEGs 13-16 revealed that, although all derivatives tested exhibited anti-tumor activity, a combination of an α-glycosidic linkage and an equatorial C-3 amine resulted in the most potent cytotoxic activity on the tested cell lines. AEG 13 having the preferred structural combination, exhibited high levels of cytotoxicity against all of the tested cell lines and is at least two orders of magnitude more potent then DOX and AE against the P-gp expressing DOX resistant ovarian cancer NAR cell line. Confocal fluorescent microscopy confirmed that AEGs maintain the permeability properties of the parent AE into anthracycline resistant tumor cells.

This study demonstrates that AEGs may serve as a promising scaffold for the development of antineoplastic agents that will overcome the widespread problem of anthracycline resistant tumors, including but not limited to tumors in which resistance is conferred by P-gp efflux pumps.

The contents of each of the references cited are incorporated by reference herein in their entirety as if fully set forth herein.

It will be appreciated by persons skilled in the art that the present invention is not limited by what has been particularly shown and described herein above. Rather the scope of the invention is defined by the claims that follow.

### REFERENCES

1. Weiss, R. B., The anthracyclines: will we ever find a better doxorubicin? Semin Oncol 1992, 19, (6), 670-86.
2. Reich, S. D.; Crooke, S. T., Anthracyclines: Current Status and New Developments. Academic Press 1980, New York.
3. Cummings, J.; Anderson, L.; Willmott, N.; Smyth, J. F., The Molecular Pharmacology of Doxorubicin Invivo. European Journal of Cancer 1991, 27, (5), 532-535.
4. Temperini, C.; Cirilli, M.; Aschi, M.; Ughetto, G., Role of the amino sugar in the DNA binding of disaccharide anthracyclines: crystal structure of the complex MAR70/d(CGATCG). Bioorg Med Chem 2005, 13, (5), 1673-9.
5. Zunino, F.; Capranico, G., DNA Topoisomerase-II as the Primary Target of Antitumor Anthracyclines. Anti-Cancer Drug Design 1990, 5, (4), 307-317.
6. Zunino, F.; Capranico, G., Sequence-selective groove binders. Cancer Therapeutics. B. Teicher Editor, Humana Press Inc, Totowa, N.J. 1997, 195-214.
7. Capranico, G.; Supino, R.; Binaschi, M.; Capolongo, L.; Grandi, M.; Suarato, A.; Zunino, F., Influence of structural modifications at the 3′ and 4′ positions of doxorubicin on the drug ability to trap topoisomerase II and to overcome multidrug resistance. Mol Pharmacol 1994, 45, (5), 908-15.
8. Binaschi, M.; Capranico, G.; DalBo, L.; Zunino, F., Relationship between lethal effects and topoisomerase II-mediated double-stranded DNA breaks produced by anthracyclines with different sequence specificity. Molecular Pharmacology 1997, 51, (6), 1053-1059.
9. Capranico, G.; Zunino, F., Antitumor inhibitors of DNA topoisomerases. Current Pharmaceutical Design 1995, 1, (1), 1-14.
10. Minotti, G.; Recalcati, S.; Mordente, A.; Liberi, G.; Calafiore, A. M.; Mancuso, C.; Preziosi, P.; Cairo, G., The secondary alcohol metabolite of doxorubicin irreversibly inactivates aconitase/iron regulatory protein-1 in cytosolic fractions from human myocardium. Faseb J 1998, 12, (7), 541-52.
11. Niitsu, N.; Kasukabe, T.; Yokoyama, A.; Okabe-Kado, J.; Yamamoto-Yamaguchi, Y.; Umeda, M.; Honma, Y., Anticancer derivative of butyric acid (pivalyloxymethyl butyrate) specifically potentiates the cytotoxicity of doxorubicin and daunorubicin through the suppression of microsomal glycosidic activity. Molecular Pharmacology 2000, 58, (1), 27-36.
12. Salvatorelli, E.; Guarnieri, S.; Menna, P.; Liberi, G.; Calafiore, A. M.; Mariggio, M. A.; Mordente, A.; Gianni, L.; Minotti, G., Defective one- or two-electron reduction of the anticancer anthracycline epirubicin in human heart—Relative importance of vesicular sequestration and impaired efficiency of electron addition. Journal of Biological Chemistry 2006, 281, (16), 10990-11001.
13. Minotti, G.; Licata, S.; Saponiero, A.; Menna, P.; Calafiore, A. M.; Di Giammarco, G.; Liberi, G.; Animati, F.; Cipollone, A.; Manzini, S.; Maggi, C. A., Anthracycline metabolism and toxicity in human myocardium: Comparisons between doxorubicin, epirubicin, and a novel disaccharide analogue with a reduced level of formation and [4Fe-4S] reactivity of its secondary alcohol metabolite. Chemical Research in Toxicology 2000, 13, (12), 1336-1341.
14. Z. Zheng, H. Aojula, D. Clarke, J Drug Target 2010, 18, 477.
15. H. J. Broxterman, K. J. Gotink, H. M. Verheul, Drug Resist Update 2009, 12, 114.
16. C. Monneret, Eur J Med Chem 2001, 36, 483.
17. E. Fan, W. Shi, T. L. Lowary, J Org Chem 2007, 72, 2917.
18. J. Gildersleeve, A. Smith, K. Sakurai, S. Raghavan, D. Kahne, Journal of the American Chemical Society 1999, 121, 6176.
19. K. Toshima, Y. Maeda, H. Ouchi, A. Asai, S. Matsumura, Bioorg Med Chem Lett 2000, 10,2163.
20. G. Zhang, L. Fang, L. Zhu, Y. Zhong, P. G. Wang, D. Sun, J Med Chem 2006, 49, 1792.
21. T. Pecere, M. V. Gazzola, C. Mucignat, C. Parolin, F. Dalla Vecchia, A. Cavaggioni, G. Basso, A. Diaspro, B. Salvato, M. Carli, G. Palu, Cancer Research 2000, 60, 2800.
22. S. O. Muller, I. Eckert, W. K. Lutz, H. Stopper, Mutat Res 1996, 371, 165.
23. B. A. P. van Gorkom, H. Timmer-Bosscha, S. de Jong, D. M. van der Kolk, J. H. Kleibeuker, E. G. E. de Vries, British Journal of Cancer 2002, 86, 1494.
24. Lear, M. J.; Yoshimura, F.; Hirama, M. A direct and efficient α-selective glycosylation protocol for the kedarcidin sugar, L-mycarose: AgPF6 as a remarkable activator of 2-deoxythioglycosides. Angewandte Chemie, International Edition. 2001, 40(5), 946-949.
25. Crich, D.; Li, W., α-selective sialylations at −78° C. in nitrile solvents with a 1-adamantanyl thiosialoside. Journal of Organic Chemistry 2007, 72, (20), 7794-7797.
26. Tietze, L. F.; G., K. M.; Guentner, C. First total synthesis of the bioactive anthraquinone kwanzoquinone C and related natural products by a Diels-Alder approach. European Journal of Organic Chemistry. 2006, (21), 4910-4915.
27. Schmidt, R. R.; Michel, J. Simple syntheses of α- and β-O-glycosyl imidates; preparation of glycosides and disaccharides. Angewandte Chemie. 1980, 92(9), 763-4.
28. Cai, S.; Stroud, M. R.; Hakomori, S.; Toyokuni, T., Synthesis of Carbocyclic Analogs of Guanosine 5′-(Beta-L-Fucopyranosyl Diphosphate) (Gdp-Fucose) as Potential Inhibitors of Fucosyltransferases. Journal of Organic Chemistry 1992, 57, (25), 6693-6696.
29. Yuasa, H.; Palcic, M. M.; Hindsgaul, O., Synthesis of the carbocyclic analog of uridine 5′-(alpha-D-galactopyranosyl diphosphate) (UDP-Gal) as an inhibitor of beta(1->4)-galactosyltransferase. Canadian Journal of Chemistry-Revue Canadienne De Chimie 1995, 73, (12), 2190-2195.
30. Ogawa, S.; Sasaki, S.; Tsunoda, H., Synthesis of Carbocyclic Analogs of the Mannosyl Trisaccharide—Ether-Linked and Imino-Linked Methyl 3,6-Bis(5a-Carba-Alpha-D-Mannopyranosyl)-3,6-Dideoxy-Alpha-D-Mannopyranosides. Carbohydrate Research 1995, 274, 183-196.
31. D'Accolti, L.; Fusco, C.; Annese, C.; Rella, M. R.; Turteltaub, J. S.; Williard, P. G.; Curci, R. Concerning the Efficient Conversion of Epoxy Alcohols into Epoxy Ketones Using Dioxiranes. Journal of Organic Chemistry. 2004, 69(24), 8510-8513.
32. Wessel, H. P., Iversen, T., and Bundle, D. R. Acid-catalysed benzylation and allylation by alkyl trichloroacetimidates. J. Chem. Soc., Perkin I, 1985, 2247-2250.33. J. C. Florent, C. Monneret, Journal of the Chemical Society-Chemical Communications 1987, 1171.
34. B. Renneberg, Y. M. Li, H. Laatsch, H. H. Fiebig, Carbohydr Res 2000, 329, 861.
35. A. Heyfets, E. Flescher, Cancer Lett 2007, 250, 300.
36. M. Liscovitch, D. Ravid, Cancer Letters 2007, 245, 350.
37. R. L. A. Furlan, L. M. Gamido, G. Brumatti, G. P. Amarante-Mendes, R. A. Martins, M. C. R. Facciotti, G. Padilla, Biotechnology Letters 2002, 24, 1807.
38. S. Mong, V. H. DuVernay, J. E. Strong, S. T. Crooke, Mol Pharmacol 1980, 17, 100.

1-51. (canceled)

52. A compound represented by the structure of formula (I) 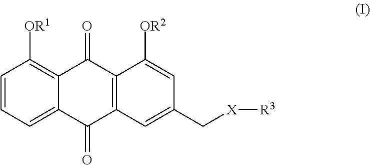
wherein
R¹ and R² are independently H or a C₁-C₄ alkyl;
R³ is an amino sugar selected from the group consisting of a 3-deoxy amino sugar, a 6-deoxy amino sugar, a 2,3-dideoxy amino sugar, a 2,6-dideoxy amino sugar, a 3,6-dideoxy amino sugar, a 2,3,6-trideoxy amino sugar, a 3-amino sugar, a 2-deoxy-3-amino sugar, a 3-deoxy-3-amino sugar, a 6-deoxy-3-amino sugar, a 2,3-dideoxy-3-amino sugar, a 2,6-dideoxy 3-amino sugar, a 3,6-dideoxy-3-amino sugar, a 2,3,6-trideoxy-3-amino sugar, a 4-deoxy amino sugar, a 2,4-deoxy amino sugar, 3,4-deoxy amino sugar, 4,6-deoxy amino sugar, a 2,3,4-trideoxy amino sugar, 3,4,6-trideoxy amino sugar, a 2,4,6-trideoxy amino sugar, a 2,3,4,6-tetradeoxy amino sugar, or a 2-deoxypyranose form of an aldopentose; or
R³ is an amino carba-sugar; and
X is O or S,
with the proviso that when both R¹ and R² are H and X is O, R³ is not 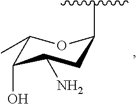
including salts, solvates, polymorphs, optical isomers, geometrical isomers, enantiomers, diastereomers, and mixtures thereof.

53. The compound of claim 52, wherein
a) R¹ and R² are H; or
b) R¹ is H and R² is CH₃; or
c) R¹ is CH₃ and R² is H; or
d) R¹ and R² are both CH₃.

54. The compound of claim 52, wherein X is O.

55. The compound of claim 52, wherein X is S.

56. The compound of claim 52, wherein R³ is an amino sugar.

57. The compound of claim 56, wherein the amino sugar is a pentose pyranoside or a hexose pyranoside selected from the group consisting of a 2-deoxypyranose form of an aldopentose, a 3-deoxypyranose form of an aldopentose, a 2,3-dideoxypyranose form of an aldopentose, a 3-aminopyranose form of an aldopentose, a 2-deoxy-3-aminopyranose form of an aldopentose, a 3-deoxy-3-aminopyranose form of an aldopentose, a 2,3-dideoxy-3-aminopyranose form of an aldopentose, a 3-deoxy pyranose form of an aldohexose, a 6-deoxy pyranose form of an aldohexose, a 2,3-dideoxy pyranose form of an aldohexose, a 2,6-dideoxy pyranose form of an aldohexose, a 3,6-dideoxy pyranose form of an aldohexose, a 2,3,6-trideoxy pyranose form of an aldohexose, a 3-aminopyranose form of an aldohexose, a 2-deoxy-3-amino-pyranose form of an aldohexose, a 3-deoxy-3-amino-pyranose form of an aldohexose, a -6-deoxy-3-amino-pyranose form of an aldohexose, a 2,3-dideoxy-3-amino-pyranose form of an aldohexose, a 2,6-dideoxy-3-amino-pyranose form of an aldohexose, a 3,6-dideoxy-3-amino-pyranose form of an aldohexose, a 2,3,6-trideoxy-3-amino-pyranose form of an aldohexose, a 4-deoxy amino pyranose form of an aldopentose, and a 4-deoxy amino pyranose form of an aldohexose.

58. The compound according to claim 56, wherein the amino sugar is a derivative of ribose, rhamnose, 2-deoxy-D or L-ribose, or 2-deoxy-D or L-rhamnose.

59. The compound of claim 56, comprising:
an amine in the equatorial position, or
an amine at the C-3 equatorial position and an α-glycosidic bond; or
an amine at the C-3 equatorial position and a β-glycosidic bond; or
an amine at the C-3 axial position and an α-glycosidic bond; or
an amine at the C-3 axial position and a β-glycosidic bond.

60. The compound of claim 56, wherein the amino sugar is selected from the group consisting of:

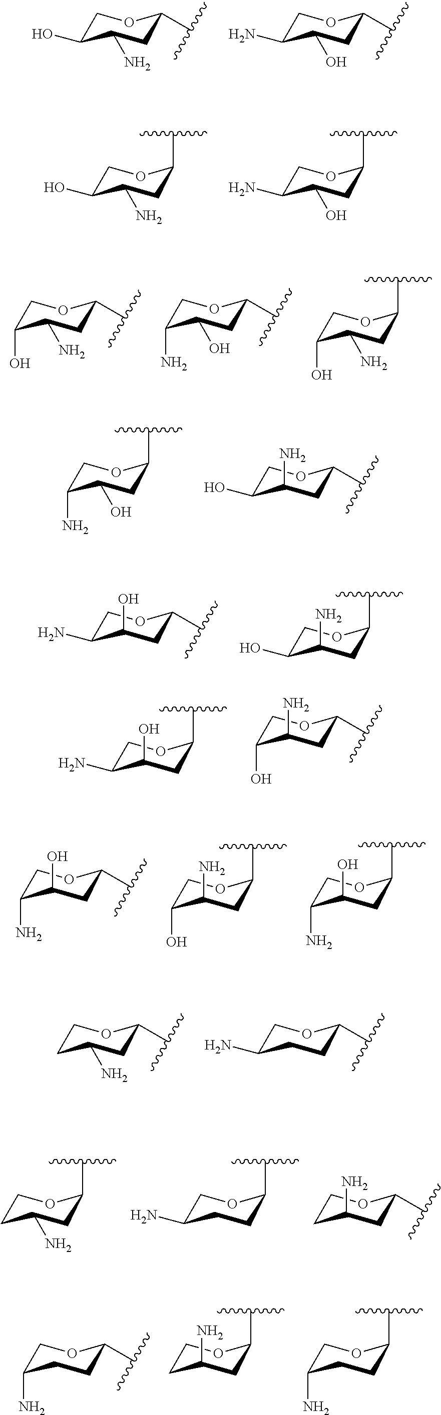

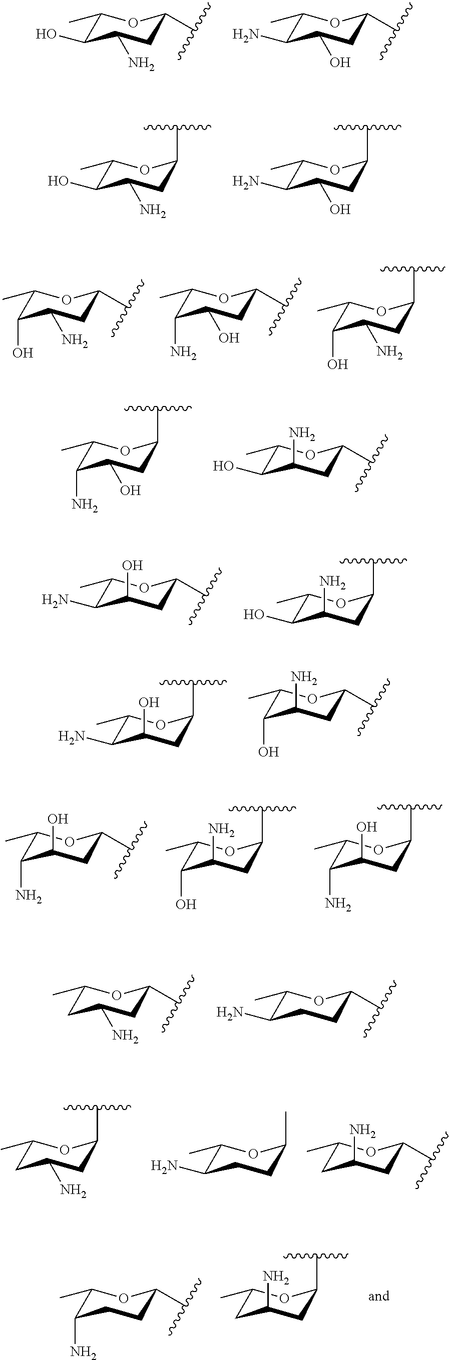

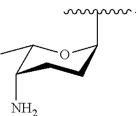

61. The compound of claim 60, wherein the amino sugar is selected from the group consisting of:

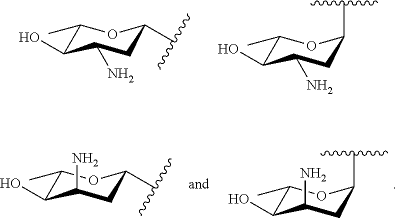

62. The compound of claim 52, which is selected from the group consisting of:

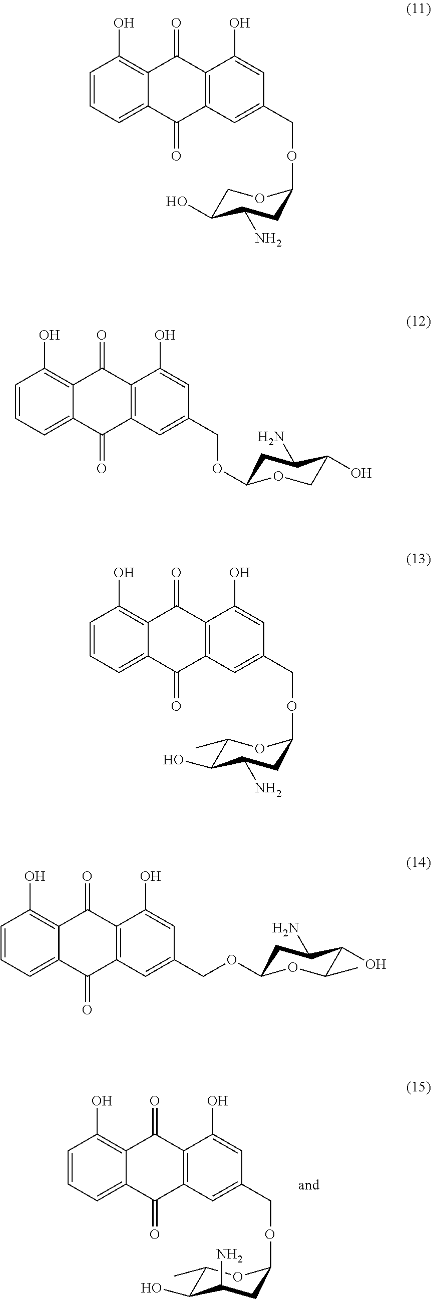

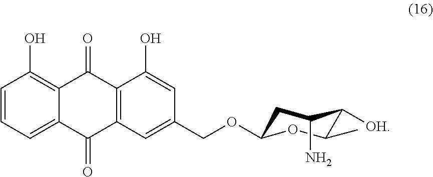

63. The compound of claim 52, wherein R³ is an amino carba-sugar.

64. The compound of claim 63, wherein the amino carba-sugar is a 2-deoxy amino carba-sugar, a 3-deoxy amino carba-sugar, a 6-deoxy amino carba-sugar, a 2,3-dideoxy amino carba-sugar, a 2,6-dideoxy amino carba-sugar, a 3,6-dideoxy amino carba-sugar, a 2,3,6-trideoxy amino carba-sugar, a 3-amino sugar, a 2-deoxy-3-amino carba-sugar, a 3-deoxy-3-amino carba-sugar, a 6-deoxy-3-amino carba-sugar, a 2,3-dideoxy-3-amino carba-sugar, a 2,6-dideoxy 3-amino carba-sugar, a 3,6-dideoxy-3-amino carba-sugar, a 2,3,6-trideoxy-3-amino carba-sugar, a 4-deoxy amino carba-sugar, a 2,4-deoxy amino carba-sugar, 3,4-deoxy amino carba-sugar, 4,6-deoxy amino carba-sugar, a 2,3,4-trideoxy amino carba-sugar, 3,4,6-trideoxy amino carba-sugar, a 2,4,6-trideoxy amino carba-sugar or a 2,3,4,6-tetradeoxy amino carba-sugar.

65. The compound of claim 63, wherein the amine group is in the equatorial position.

66. The compound of claim 63, wherein the amino carba-sugar is selected from the group consisting of:

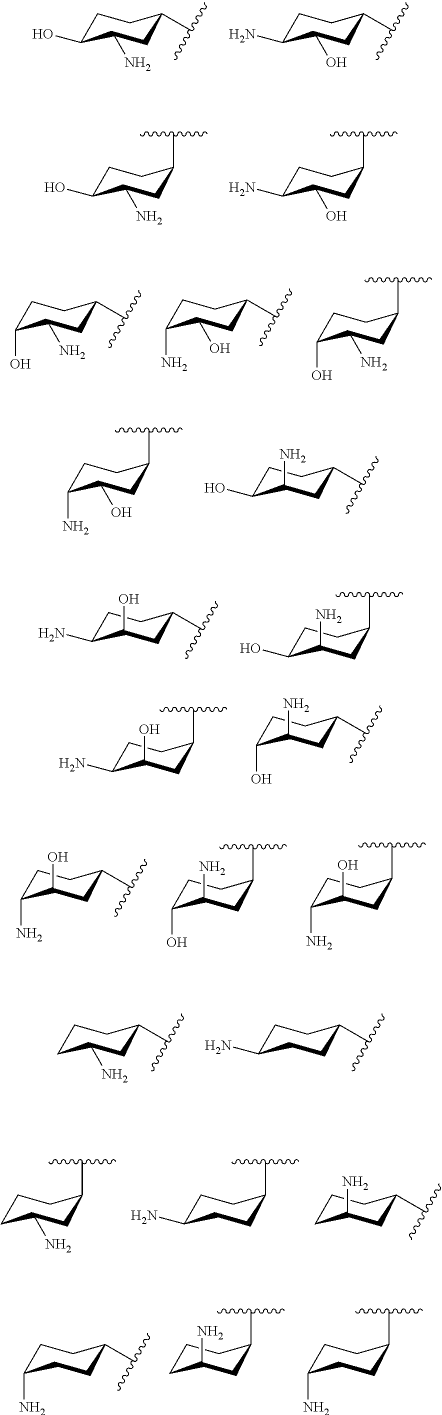

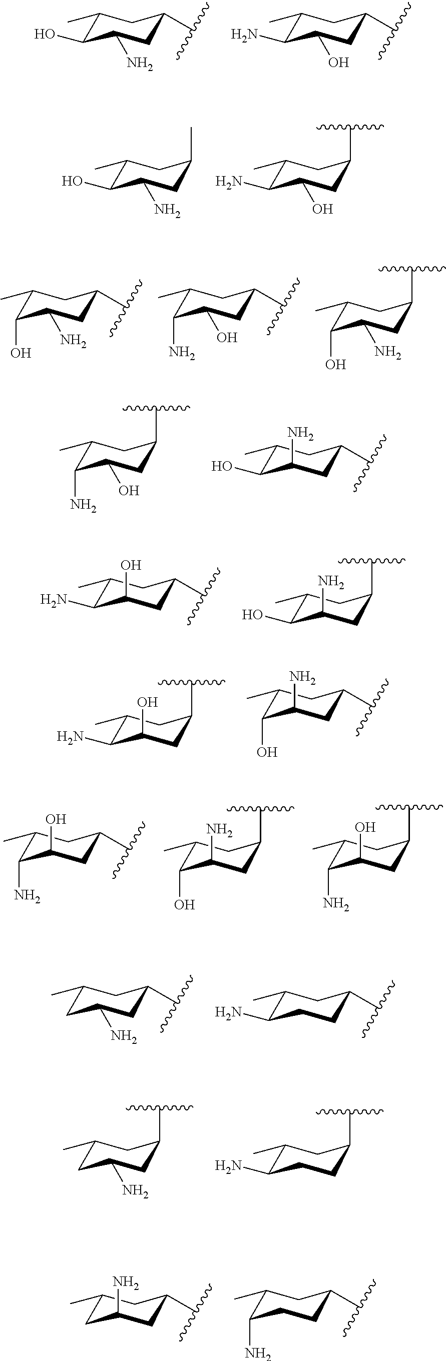

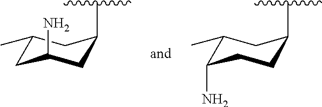

67. The compound of claim 63, which is represented by the structure:

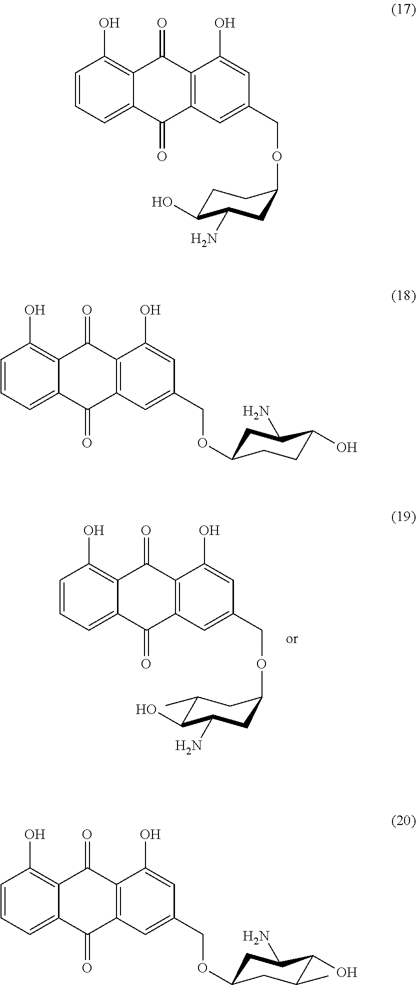

68. A pharmaceutical composition comprising a compound of claim 52, and a pharmaceutically acceptable excipient.

69. The pharmaceutical composition of claim 68, wherein the composition is in a form suitable for oral administration, intravenous administration by injection, topical administration, administration by inhalation, or administration via a suppository.

70. A method for inhibiting cancer cell proliferation, comprising contacting said cancer cell with a therapeutically effective amount of a compound according claim 52, or a pharmaceutical composition comprising such compound.

71. A method of treating cancer in a subject in need thereof, comprising the step of administering to the subject a therapeutically effective amount of a compound according to claim 52, or a pharmaceutical composition comprising such compound.

72. The method of claim 71, wherein the cancer is selected from the group consisting of lymphoproliferative disorders, breast cancer, ovarian cancer, prostate cancer, cervical cancer, endometrial cancer, bone cancer, liver cancer, stomach cancer, colon cancer, pancreatic cancer, cancer of the thyroid, head and neck cancer, cancer of the central nervous system, cancer of the peripheral nervous system, skin cancer, kidney cancer, hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, squamous cell carcinoma, basal cell carcinoma, adenocarcinoma (well differentiated, moderately differentiated, poorly differentiated or undifferentiated), renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, T-cell leukemia, multiple myeloma, myeloid lymphoma, Hodgkin's lymphoma and non-Hodgkin's lymphoma, as well as metastases of all of the above.

73. The method of claim 71, wherein the cancer is characterized by resistance to anthracycline chemotherapeutic agents.

74. The method of claim 73, wherein the cancer is characterized by resistance to doxorubicin.

75. The method of claim 73, wherein the cancer is selected from the group consisting of lymphoproliferative disorders, breast cancer, ovarian cancer, prostate cancer, colon cancer, pancreatic cancer, sarcomas, fibrosarcoma, melanoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma and non-Hodgkin's lymphoma, as well as metastases of all of the above.

76. The method of claim 73, wherein resistance is conferred by overexpression of P-gp efflux pumps.

77. A process for preparing compound represented by the structure of formula (I) according to claim 52, comprising the step of coupling a compound of formula (II) 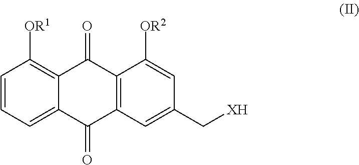
or an activated derivative thereof, optionally in the presence of a catalyst, with an amino sugar or amino carba-sugar derivative of formula R³—Y wherein Y is a leaving group and R¹, R², R³ and X are as defined in claim 52.

78. The process according to claim 77, wherein R³ is an amino sugar, and the process comprises the following steps:
(i) coupling a compound of formula (II) or an activated derivative thereof, optionally in the presence of a catalyst, with an amino sugar derivative represented by the structure of formula (III): 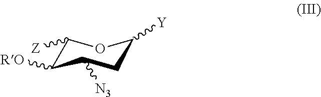
wherein Y is a leaving group, R′ is a hydroxyl protecting group, Z is H or CH₃, wherein the substituents Z, OR′, N₃ and Y can each be in the equatorial or axial position, so as to generate a compound of formula (IV): 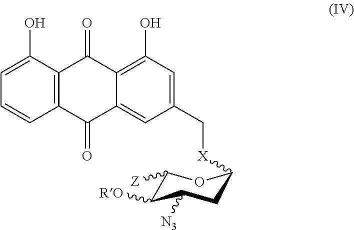
(ii) removing the hydroxy protecting group R′ to generate a free hydroxyl; and
(iii) converting the azide group (N₃) to an amine (NH₂);
wherein steps (ii) and (iii) can be conducted in any order.
